(19) United States
(12) Patent Application Publication
(10) Pub. No.: US 2023/0279477 A1
(43) Pub. Date: Sep. 7, 2023

(54) METHODS FOR SPATIAL ANALYSIS USING TARGETED RNA CAPTURE

(71) Applicant: 10x Genomics, Inc., Pleasanton, CA (US)

(72) Inventors: Linda Kvastad, Huddinge (SE); Mohammadreza Mirzazadeh, Stockholm (SE); Ludvig Ale Larsson, Stockholm (SE); Joakim Lundeberg, Stockholm (SE)

(21) Appl. No.: 18/302,443

(22) Filed: Apr. 18, 2023

Related U.S. Application Data
(63) Continuation of application No. PCT/US2022/047835, filed on Oct. 26, 2022.
(60) Provisional application No. 63/271,754, filed on Oct. 26, 2021.

(51) Int. Cl. C12Q 1/6841; C12Q 1/6876; C12Q 1/686

(52) U.S. Cl. CPC C12Q 1/6841; C12Q 1/6876; C12Q 1/686; C12Q 2600/178

(57) ABSTRACT

Provided herein are methods for spatial analysis that captures non-poly(A)-containing RNA molecules, such as long non-coding RNAs and microRNAs. Methods, kits, and compositions for spatial analysis using targeted RNA capture using randomer capture probes are disclosed herein.

### CROSS-REFERENCE TO RELATED APPLICATIONS

Pursuant to 35 U.S.C. § 119(e), this application is a continuation of International Application PCT/US2022/047835, with an international filing date of Oct. 26, 2022, which claims priority to U.S. Provisional Application Serial No. 63/271,754, filed on Oct. 26, 2021. The disclosure of the prior application is considered part of the disclosure of this application and is incorporated herein by reference in its entirety.

### SEQUENCE LISTING

This application contains a Sequence Listing that has been submitted electronically as an XML file named “47706-0313001_SequenceListing.XML.” The XML file, created on Mar. 29, 2023, is 2,318 bytes in size. The material in the XML file is hereby incorporated by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Long noncoding RNAs (lncRNAs) and microRNAs (miRNAs) (i.e., RNAs not comprising a poly(A) tail) constitute a considerable proportion of the total RNA pool from the biological sample. However, because of this high proportion, these RNA molecules often interfere with methods of spatial analysis, complicating the goal of studying both analytes of interest having a poly(A) tail and analytes such as lncRNAs and miRNAs, lacking poly(A) tails. Thus, there remains a need to develop spatial methods to efficiently and accurately determine the location and abundance of each of these RNA groups.

### SUMMARY

The present disclosure relates to methods of capturing RNA molecules lacking a poly(A) tail from a biological sample comprising nucleic acid molecules. Applicant has identified that distribution of capture probes comprising randomer sequences aids in detection of RNA molecules lacking a poly(A) tail. This technology, in some instances, can be combined with technology that detects RNA molecules having a poly(A) tail (e.g., mRNA). Accordingly, the disclosure is useful for determining the location and abundance of multiple types of RNA molecules from fixed paraffin embedded (FFPE) or fresh tissue samples, for spatial analysis of desirable analytes.

Thus, one features disclosed herein is a method for determining abundance and location of an RNA molecule lacking a poly(A) sequence in a biological sample. In some instances, the method comprises (a) placing the biological sample onto an array, wherein the array comprises a plurality of randomer capture probes, wherein a randomer capture probe of the plurality of randomer capture probes comprises a sequences that is substantially complementary to all or a portion to a sequence of the RNA molecule lacking the poly(A) tail; (b) hybridizing the randomer capture probe to the RNA molecule lacking the poly(A) sequence; and (c) determining (i) all or part of the sequence of the RNA molecule lacking the poly(A) sequence bound to the randomer capture probe to determining the abundance and the location of the RNA molecule lacking the poly(A) sequence in the biological sample.

In some instances, the randomer capture probe is a DNA probe. In some instances, the randomer capture probe comprises a random hexamer sequence. In some instances, the randomer capture probe comprises a random nonomer sequence. In some instances, the randomer capture probe comprises one or more modified nucleotides. In some instances, the modified nucleotides are locked nucleic acids. In some instances, the randomer capture probe further includes a randomer spatial barcode. In some instances, the RNA molecule lacking the poly(A) sequence is a long noncoding RNA (lncRNA). In some instances, the RNA molecule lacking the poly(A) sequence is a microRNA (miRNA). In some instances, the RNA molecule lacking a poly(A) sequence is a small interfering RNA (siRNA) molecule, a Piwi-interacting RNA (piRNA) molecule, a small nucleolar RNA (snoRNA) molecule, or a long intervening/intergenic noncoding RNAs (lincRNA) molecule.

In some instances, the methods of the above embodiments further comprise providing a plurality of undesirable RNA depletion probes to the biological sample, thereby generating a plurality of undesirable RNA depletion probe-undesirable RNA complexes, wherein an undesirable RNA depletion probe of the plurality of undesirable RNA depletion probes is substantially complementary to a sequence of an undesirable RNA molecule in the biological sample. In some instances, providing the plurality of undesirable RNA depletion probes to the biological sample is performed between steps (a) and (b) in the embodiments above. In some instances, the undesirable RNA depletion probe is a DNA probe. In some instances, the undesirable RNA molecule is a transfer RNA (tRNA), a ribosomal RNA (rRNA), a messenger RNA (mRNA), or any combinations thereof. In some instances, the undesirable RNA molecule is a mitochondrial RNA, nuclear RNA, or cytoplasmic RNA. In some instances, at least one undesirable RNA depletion probe specifically hybridizes to substantially the entire full length sequence of the undesirable RNA molecule. In some instances, the undesirable RNA depletion probe is substantially complementary to all or a portion of the sequence of the undesirable RNA molecule in the biological sample.

In some instances, the methods of any of the embodiments above further include removing the plurality of undesirable RNA depletion probe-undesirable RNA complexes to deplete the undesirable RNA molecules prior to hybridizing the RNA molecule lacking the poly(A) sequence to the randomer capture probe affixed to the substrate. In some instances, at least one undesirable RNA depletion probe specifically hybridizes to substantially one or more portions of the sequence of the undesirable RNA molecule. In some instances, the removing step comprises contacting the undesirable RNA depletion probe with a ribonuclease. In some instances, the ribonuclease is RNase H. In some instances, the RNase H is RNase H1, RNase H2, and/or a thermostable RNase.

In some instances, the undesirable RNA depletion probe further comprises a capture moiety, wherein the removing step comprises using a capture moiety-binding agent that binds specifically to the capture moiety. In some instances, the capture moiety is streptavidin, avidin, biotin, or a fluorophore. In some instances, the capture moiety is a biotin. In some instances, the capture moiety comprises a small molecule, a nucleic acid, or a carbohydrate. In some instances, the capture moiety is positioned 5′ or 3′ to the domain in the undesirable RNA depletion probe.

In some instances, a capture moiety-binding agent that binds specifically to the capture moiety comprises a protein. In some instances, the protein is an antibody. In some instances, the protein is streptavidin. In some instances, the capture moiety-binding agent that binds specifically to the capture moiety comprises a nucleic acid. In some instances, the nucleic acid is DNA. In some instances, the capture moiety-binding agent that binds specifically to the capture moiety comprises a small molecule. In some instances, the capture moiety-binding agent that binds specifically to the capture moiety is attached to a substrate. In some instances, the substrate is a bead. In some instances, the bead is a magnetic bead. In some instances, the capture moiety is a biotin and the capture moiety-binding agent is streptavidin, wherein the streptavidin is attached to a magnetic bead that allows the undesirable RNA depletion probe-undesirable RNA complexes to be removed magnetically from the biological sample.

In some instances, the methods of any one of the embodiments described herein includes methods comprising a biological sample that was previously stained. In some instances, the biological sample was previously stained using hematoxylin and eosin (H&E). In some instances, the biological sample was previously stained using immunofluorescence or immunohistochemistry. In some instances, the method further comprises contacting the biological sample with a permeabilization agent. In some instances, the biological sample is permeabilized with a permeabilization agent. In some instances, the permeabilization agent is selected from an organic solvent, a detergent, and an enzyme, or a combination thereof. In some instances, the permeabilization agent is an endopeptidase or protease. In some instances, the endopeptidase is pepsin. In some instances, the endopeptidase is proteinase K.

In some instances, the methods of any of the embodiments above include extending a 3′ end of the randomer capture probe using the RNA molecule lacking the poly(A) tail that is bound to the randomer capture domain as a template to generate an extended randomer capture probe;

In some instances, the methods of any of the embodiments above include amplifying the extended randomer capture probe prior to step (c), thereby generating an amplified product. In some instances, the amplified product comprises (i) all or part of sequence of the randomer capture probes or a complement thereof, (ii) all or a part of the sequence of the RNA molecule lacking the poly(A) tail, or a complement thereof, and (iii) the randomer spatial barcode, or a complement thereof. In some instances, the determining step comprises sequencing.

In some instances, the RNA molecule lacking the poly(A) sequence is associated with a disease or condition. In some instances, the biological sample is a tissue sample. In some instances, the tissue sample is a formalin-fixed, paraffin-embedded (FFPE) tissue sample, a fresh tissue sample, or a frozen tissue sample. In some instances, the tissue sample is the FFPE tissue sample, and the tissue sample is decrosslinked.

In some instances, the methods of any of the above embodiments further include modifications to the array. In some instances, the array further comprises a plurality of capture probes, wherein a capture probe of the plurality of capture probes comprises a spatial barcode and a capture domain. In some instances, the capture domain comprises a poly-thymine sequence. In some instances, the capture probes and the randomer capture probes are distributed substantially evenly on the array, and/or wherein concentration of the capture probes and concentration of the randomer capture probes on the array is substantially the same. In some instances, concentration of the capture probes one the array is higher than concentration of the randomer capture probes on the array, or wherein concentration of the capture probes one the array is lower than concentration of the randomer capture probes on the array.

In some instances, the methods of any of the above embodiments further include detecting abundance and location of an analyte in a biological sample using a templated ligation. In some instances, the methods comprise: after step (a), contacting a biological sample with a first templated ligation (RTL) probe, a second RTL probe, wherein the first RTL probe and the second RTL probe are substantially complementary to adjacent sequences of the analyte, and wherein the second RTL probe comprises a capture probe binding domain that is capable of binding to a capture domain; hybridizing the first RTL probe and the second RTL probe to the analyte; ligating the first RTL probe and the second RTL probe, thereby creating a ligated probe that is substantially complementary to the analyte; releasing the ligated probe from the analyte; hybridizing the capture probe binding domain to a capture domain; and determining (i) all or a part of the sequence of the ligated probe specifically bound to the capture domain, or a complement thereof, and (ii) the sequence of the spatial barcode, or a complement thereof, and using the determined sequence of (i) and (ii) to identify the location of the analyte in the biological sample. In some instances, the first RTL probe comprises at least two ribonucleic acid bases at the 3′ end. In some instances, the first RTL probe further comprises a functional sequence. In some instances, the functional sequence is a primer sequence. In some instances, the second RTL probe comprises a phosphorylated nucleotide at the 5′ end.

In some instances, the method further comprises providing a capture probe binding domain blocking moiety that interacts with the capture probe binding domain. In some instances, the method further comprises releasing the capture probe binding domain blocking moiety from the capture probe binding domain prior to hybridizing the capture probe binding domain to a capture domain. In some instances, the capture probe binding domain comprises a poly-adenylated (poly(A)) sequence, or a complement thereof. In some instances, the capture probe binding domain blocking moiety comprises a poly-uridine sequence, a poly-thymidine sequence, or both. In some instances, releasing the poly-uridine sequence from the poly(A) sequence comprises denaturing the ligated probe or contacting the ligated probe with an endonuclease or exonuclease. In some instances, the capture probe binding domain comprises a sequence that is complementary to all or a portion of the capture domain of the capture probe. In some instances, the capture probe binding domain comprises a degenerate sequence. In some instances, the ligation step comprises ligating the first and second RTL probe using enzymatic ligation or chemical ligation. In some instances, the enzymatic ligation utilizes a ligase. In some instances, the ligase is one or more of a T4 RNA ligase (Rnl2), a splintR ligase, a single stranded DNA ligase, or a T4 DNA ligase. In some instances, the ligase is a T4 RNA ligase 2 (Rnl2) ligase.

In some instances, the first RTL probe and the second RTL probe are DNA probes. In some instances, hybridizing the first RTL probe and the second RTL probe to the analyte generates a RNA: DNA hybrid. In some instances, releasing the ligated probe from the analyte comprises contacting the ligated probe with a ribonuclease. In some instances, the ribonuclease is RNase H. In some instances, the RNase H is RNase H1, RNase H2, or a thermostable RNase.

In some instances, the method of any of the above embodiments further comprises amplifying the ligated probe prior to determining (i) all or a part of the sequence of the ligated probe specifically bound to the capture domain, or a complement thereof, and (ii) the sequence of the spatial barcode, or a complement thereof. In some instances, determining (i) all or a part of the sequence of the ligated probe specifically bound to the capture domain, or a complement thereof, and (ii) the sequence of the spatial barcode, or a complement thereof, comprises sequencing.

In some instances, the analyte is RNA. In some instances, the RNA is an mRNA. In some instances, the methods of any of the above embodiments include detecting abundance and location of an analyte in a biological sample by the steps of: hybridizing the analyte to the capture probe; and determining (i) all or a part of a sequence corresponding to the analyte, or a complement thereof, and (ii) the spatial barcode, or a complement thereof, and using the determined sequence of (i) and (ii) to identify the abundance and location of the analyte in the biological sample. In some instances, after hybridizing the analyte to the capture probe, extending the capture probe using the analyte as a template, thereby generating an extended capture probe. In some instances, the methods further include amplifying the extended capture probe. In some instances, the methods further include determining (i) all or a part of a sequence corresponding to the analyte, or a complement thereof, and (ii) the spatial barcode, or a complement thereof, comprises sequencing.

In some instances, the methods of any of the above embodiments further include detecting abundance and location of an analyte in a biological sample by the steps of: attaching the biological sample with a plurality of analyte capture agents, wherein an analyte capture agent of the plurality of analyte capture agents comprises: (i) an analyte binding moiety that binds specifically to the analyte; (ii) an analyte binding moiety barcode; and (iii) an analyte capture sequence, wherein the analyte capture sequence binds specifically to the capture domain; hybridizing the analyte capture sequence to the capture probe; and determining (i) all or a part of a sequence of the analyte capture sequence, or a complement thereof, and (ii) the spatial barcode, or a complement thereof, and using the determined sequence of (i) and (ii) to identify the abundance and location of the analyte in the biological sample. In some instances, after hybridizing the analyte capture sequence to the capture probe, extending the capture probe using the analyte capture sequence as a template, thereby generating an extended capture probe. In some instances, the methods further include amplifying the extended capture probe. In some instances, the methods further include determining (i) all or a part of a sequence of the analyte capture sequence, or a complement thereof, and (ii) the spatial barcode, or a complement thereof, comprises sequencing.

Also featured herein are spatial arrays. In some instances, the spatial arrays include: a plurality of randomer capture probes, wherein a randomer capture probes of the plurality of randomer capture probes is capable of hybridizing to the RNA molecule lacking a poly(A) tail; and wherein the randomer capture probe comprises a random hexamer sequence or a random nonomer sequence; and a plurality of capture probes, wherein a capture probe of the plurality of capture probes comprises a spatial barcode and a capture domain. In some instances, the randomer capture probe further comprises one or more randomer functional domains, a randomer unique molecular identifier, a randomer cleavage domain, and combinations thereof. In some instances, the randomer capture probe is a DNA probe. In some instances, the randomer capture probe comprises one or more modified nucleotides. In some instances, the modified nucleotides are locked nucleic acids. In some instances, the capture domain comprises a poly-thymine sequence. In some instances, the capture probes and the randomer capture probes are distributed substantially evenly on the array, and/or wherein concentration of the capture probes and concentration of the randomer capture probes on the array is substantially the same. In some instances, concentration of the capture probes one the array is higher than concentration of the randomer capture probes on the array. In some instances, concentration of the capture probes one the array is lower than concentration of the randomer capture probes on the array. In some instances, the capture probe further comprises one or more functional domains, a unique molecular identifier, a cleavage domain, and combinations thereof.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2A shows an exemplary graph quantifying the total genes captured when practicing different RNA capture scenarios for spatial gene analysis using randomers in mouse olfactory bulb tissue sections.

FIG. 2B shows an exemplary companion graph for FIG. 2A quantifying the UMI counts detected in the mouse olfactory bulb tissue sections when practicing different RNA capture scenarios using randomers.

FIG. 3A shows an exemplary graph reporting the percent mt-Rnr1 captured when practicing the different RNA capture scenarios in mouse olfactory bulb tissue sections from FIGS. 2A and 2B.

FIG. 3B shows an exemplary graph reporting the percent mt-Rnr2 captured when practicing the different RNA capture scenarios using randomers in mouse olfactory bulb tissue sections.

FIG. 4 shows the unique genes and biotype proportions for the targets of randomer capture probes (i.e., nonomers, hexamers), poly(T) negative control sequences, RD probes, and combinations thereof.

FIGS. 5A and 5B shows graphs quantifying the total genes and total UMIs when a mouse brain sample targets were captured using only nonomer capture probes or only hexamer capture probes.

FIG. 6 shows spatial expression of Snhg14 in two different mouse brain samples.

FIG. 7 shows enhanced spatial expression detection with a locked nucleic acid (LNA) nonomer capture probe (9N-v1_Tm48.6_LNA) at 48.6° C. (left) compared to a control nonomer capture probe (right).

### DETAILED DESCRIPTION

### I. Introduction

Disclosed herein are methods and compositions predicated on using targeted RNA capture to detect one or more species of RNA molecules lacking a poly(A) tail (e.g., lncRNA or miRNA) either independently or in combination with other spatial methods (e.g., mRNA capture, templated ligation, RNA depletion). To achieve capture of RNA molecules lacking poly(A) tails, one or more randomer capture probes are designed that hybridize to RNA molecules lacking poly(A) tails. For example, in one embodiment, capture probes can be affixed to a substrate that selectively hybridize to lncRNA in a biological sample. RNA capture can be combined with spatial analysis techniques in order to determine abundance and/or location of one or more analytes in a biological sample. The ability to detect both a target of interest (e.g., using RTL, direct mRNA capture) along with detecting one or more RNA molecules lacking a poly(A) tail increases the amount of data and information, efficiency and sensitivity of the spatial analysis techniques.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 20 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, PCT Publ. Nos. WO 2018/091676, WO 2020/176788, WO 2022/140028, Rodrigues etal., Science 363(6434):1463-1467, 2019; Lee etal., Nat. Protoc. 10(3):442-458, 2015; Trejo etal., PLoS ONE 14(2):e0212031, 2019; Chen etal., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10x Genomics Support Documentation website, and can be used herein in any combination.

Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

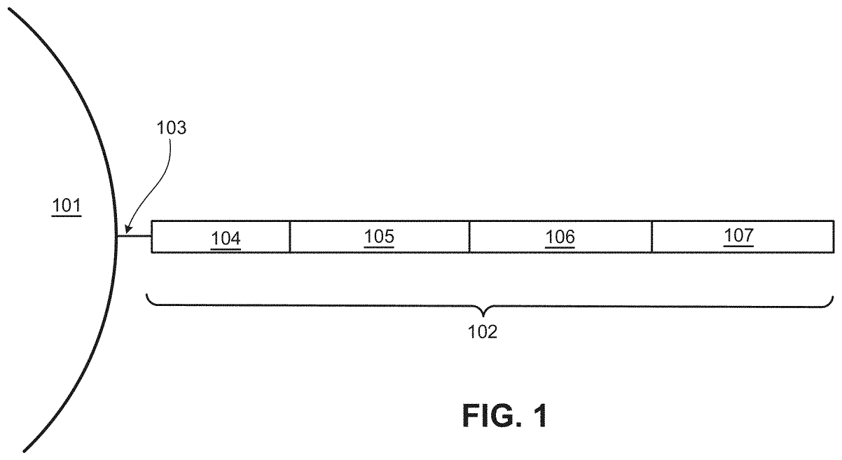

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

Additional capture embodiments can include a cleavable capture probe, wherein the cleaved capture probe can enter into a non-permeabilized cell and bind to analytes within the sample. In some instances, the capture probe contains a cleavage domain, a cell penetrating peptide, a reporter molecule, and a disulfide bond (—S—S—). In some instances, the capture probe also includes a spatial barcode and a capture domain.

In another embodiment, capture probes can be multiplexed to comprise exemplary multiplexed spatially-barcoded feature. In some instances, the feature can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode. One type of capture probe associated with the feature includes the spatial barcode in combination with a poly(T) capture domain, designed to capture mRNA target analytes. A second type of capture probe associated with the feature includes the spatial barcode in combination with a random N-mer capture domain for RNA or gDNA analysis, such as the randomer capture domains described herein. A third type of capture probe associated with the feature includes the spatial barcode in combination with a capture domain complementary to a capture handle sequence of an analyte capture agent of interest. A fourth type of capture probe associated with the feature includes the spatial barcode in combination with a capture domain that can specifically bind a nucleic acid molecule that can function in a CRISPR assay (e.g., CRISPR/Cas9). It is appreciated that capture-probe barcoded constructs can be tailored for analyses of any given analyte associated with a nucleic acid and capable of binding with such a construct. For example, these schemes can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

In some instances, an exemplary analyte capture agent comprises an analyte-binding moiety and an analyte-binding moiety barcode domain. The exemplary analyte -binding moiety is a molecule capable of binding to an analyte and the analyte capture agent is capable of interacting with a spatially-barcoded capture probe. The analyte -binding moiety can bind to the analyte with high affinity and/or with high specificity. The analyte capture agent can include an analyte -binding moiety barcode domain, a nucleotide sequence (e.g., an oligonucleotide), which can hybridize to at least a portion or an entirety of a capture domain of a capture probe. The analyte-binding moiety barcode domain can comprise an analyte binding moiety barcode and a capture handle sequence described herein. The analyte-binding moiety can include a polypeptide and/or an aptamer. The analyte-binding moiety can include an antibody or antibody fragment (e.g., an antigen-binding fragment).

During an interaction between a feature-immobilized capture probe and an analyte capture agent. The feature-immobilized capture probe can include a spatial barcode as well as functional sequences and UMI, as described elsewhere herein. The capture probe can also include a capture domain that is capable of binding to an analyte capture agent. The analyte capture agent can include a functional sequence, analyte binding moiety barcode, and a capture handle sequence that is capable of binding to the capture domain of the capture probe. The analyte capture agent can also include a linker that allows the capture agent barcode domain to couple to the analyte binding moiety.

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction). Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using templated ligation. Methods of templated ligation have been described previously. See, e.g., Credle et al., Nucleic Acids Res. 2017 Aug. 21;45(14):e128. Typically, templated ligation includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease (e.g., RNAse H). The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application Publ. No. WO 2021/102003 and/or U.S. Patent Application Publication No. US 2021/0155982 A1.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application Publ. No. WO 2021/067514 and spatial analysis methods are generally described in WO 2020/061108 and/or U.S. Patent Application Publication No. US 2021/0155982 A1.

In some embodiments of a method disclosed herein, one or more analytes from the biological sample are released from the biological sample and migrate to a substrate comprising an array of capture probes for attachment to the capture probes of the array, either directly or indirectly. In some embodiments, the release and migration of the analytes to the substrate comprising the array of capture probes occurs in a manner that preserves the original spatial context of the analytes in the biological sample. In some embodiments, the biological sample is mounted on a first substrate and the substrate comprising the array of capture probes is a second substrate. In some embodiments, the method is facilitated by a sandwiching process. Sandwiching processes are described in, e.g., US. Patent Application Pub. No. 20210189475, PCT Publ. Nos. WO 2021/252747, WO 2022/051152, and WO 2022/140028, each of which is incorporated by reference in its entirety. In some embodiments, the sandwiching process may be facilitated by a device, sample holder, sample handling apparatus, or system described in, e.g., US. Patent Application Pub. No. 20210189475, WO 2021/252747, or WO 2022/061152.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., slide) is in a superior position to the first substrate (e.g., slide). In some embodiments, the first substrate (e.g., slide) may be positioned superior to the second substrate (e.g., slide). A reagent medium within a gap between the first substrate (e.g., slide) and the second substrate (e.g., slide) creates a liquid interface between the two substrates. The reagent medium may be a permeabilization solution which permeabilizes and/or digests the sample. In some embodiments wherein the sample has been pre-permeabilized, the reagent medium is not a permeabilization solution. In some embodiments, analytes (e.g., mRNA transcripts) and/or intermediate agents of the biological sample may release from the biological sample, actively or passively migrate (e.g., diffuse) across the gap toward the capture probes, and bind on the capture probes. In some embodiments, the active migration is via electrophoresis. Electrophoretic migration methods are further described in US. Patent Application Pub. No. 20210189475, which is hereby incorporated by reference.

In some instances, one or more spacers may be positioned between the first substrate (e.g., slide) and the second substrate (e.g., slide including spatially barcoded capture probes). The one or more spacers may be configured to maintain a separation distance between the first substrate and the second substrate. While the one or more spacers is shown as disposed on the second substrate, the spacer may additionally or alternatively be disposed on the first substrate.

In some embodiments, the one or more spacers is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application Publ. No. WO 2021/102005, and/or U.S. Patent Application Publ. No. US 2021/0158522 A1. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Detection of RNA Molecules Lacking Poly(A) Tails Using Randomer Capture Probes

RNA capture traditionally takes advantage of hybridization of a capture domain comprising a polythymidated sequence to a poly(A) tail of an mRNA molecule. However, this approach misses detection of RNA molecules lacking a poly(A) tail, including, but not limited to, long non-coding RNAs (lncRNAs) and microRNAs (miRNAs). To address this issue, provided herein is a substrate comprising one or more randomer capture domains of capture probes (e.g., hexamers or nonomers) that are designed to hybridize to one or more RNA molecules lacking a lacking a poly(A) tail (e.g., long noncoding RNAs (lncRNAs)). For example, in one embodiment, randomer capture probe domains are designed that selectively hybridize to lncRNAs in a biological sample, and these “randomer capture probe domains” can be used to detect the presence and abundance of RNA molecules lacking a poly(A) tail (e.g., lncRNAs). In some instances disclosed herein, randomer capture probes are generated on a spatial array using splint oligonucleotide technology. In some instances, the randomer capture probes are generated by (a) providing an array comprising a plurality of oligonucleotides, wherein the 3′ end of an oligonucleotide of the plurality of oligonucleotides is attached to a substrate; (b) providing a plurality of primers, wherein a primer of the plurality of primers is substantially complementary to a portion of the oligonucleotide; (c) extending the primer using the oligonucleotide as a template, thereby generating a first oligonucleotide with a free 3′ end; (d) extending the first oligonucleotide to produce a 3′ overhang; (e) providing a splint oligonucleotide that hybridizes to the 3′ end of the first oligonucleotide; and (f) ligating a randomer capture probe to the 3′ end of the first oligonucleotide, thereby generating a spatial array.

In one embodiment, disclosed herein is a method for determining abundance and location of an RNA molecule lacking a poly(A) sequence in a biological sample. In some instances, the method comprises (a) placing the biological sample onto an array, wherein the array comprises a plurality of randomer capture probes (e.g., generated using splint oligonucleotide technology as described above), wherein a randomer capture probe of the plurality of randomer capture probes comprises a sequence that is substantially complementary to all or a portion of a sequence of the RNA molecule lacking the poly(A) tail; (b) hybridizing the randomer capture probe to the RNA molecule lacking the poly(A) sequence; and (c) determining (i) all or part of the sequence of the RNA molecule lacking the poly(A) sequence bound to the randomer capture probe to determine and correlate the abundance and the location of the RNA molecule lacking the poly(A) sequence in the biological sample.

The methods of determining the abundance and the location of the RNA molecule lacking the poly(A) sequence can be combined with other embodiments of the disclosure. For instance, RNA depletion probes can be provided (e.g., added to) to the biological sample during the methods of determining the abundance and the location of the RNA molecule lacking the poly(A) sequence via capture of these RNA molecules using randomer capture probes.

A further non-limiting example of a method for identifying a location of an analyte in a biological sample using the combination of RNA-templated ligation and randomer probe capture (with and without the embodiment of providing RNA depletion probes). In these instances, arrays used for this method have a combination of randomer capture probes and poly(T) comprising capture probes. The method herein includes: (a) contacting the biological sample with a substrate, wherein the substrate comprises (i) a plurality of capture probes, each comprising a capture domain and optionally a spatial barcode and (ii) a plurality of randomer capture probes, each comprising a sequence that is substantially complementary to a sequence of an RNA molecule lacking a poly(A) tail in the biological sample; (b) contacting a biological sample with a first templated ligation (RTL) probe, a second RTL probe, wherein the first RTL probe and the second RTL probe are substantially complementary to adjacent sequences of the analyte, wherein the second RTL probe comprises a capture probe binding domain that is capable of binding to a capture domain of a capture probe affixed to a substrate, wherein the capture probe further comprises a spatial barcode; (c) hybridizing the first RTL probe and the second RTL probe to the analyte; (d) ligating the first RTL probe and the second RTL probe, thereby generating a ligation product that is substantially complementary to the analyte; (e) hybridizing (i) the capture probe binding domain of the ligation product to a capture domain of a capture probe on the substrate and (ii) the randomer capture probe to the RNA molecule lacking a poly(A) tail; (f) determining (i) all or a part of the sequence of the ligation product bound to the capture domain, or a complement thereof, and (ii) the spatial barcode, or a complement thereof, and using the determined sequence of (i) and (ii) to identify the location of the analyte in the biological sample; and (g) determining the abundance and location of the RNA molecule lacking a poly(A) tail in the biological sample.

### (a) RNA Molecule(s) Lacking Poly(A) Tails

As used herein, the term “RNA molecule lacking a poly(A) tail” refers to an RNA molecule that does not have a poly(A) tail at its 3′ end. For comparison, eukaryotic mRNA typically includes a 3′ poly(A) tail.

In some embodiments, the RNA molecule lacking a poly(A) tail includes long noncoding RNAs. Long non-coding RNAs (also abbreviated as long ncRNAs or lncRNAs throughout) are a type of RNA transcript with lengths exceeding 200 nucleotides that are not translated into protein. Traditionally viewed as transcriptional noise, they have emerged as important regulators of cellular functions such as protein synthesis, RNA maturation/transport, chromatin remodeling, and transcriptional activation and/or repression programs. See St Laurent et al., Trends Genet. (2015) 31(5):239-51, and Kung et al., Genetics. (2013) 193(3):651-69, each of which is incorporated by reference in its entirety. lncRNAs have been shown to influence biological processes such as stem cell pluripotency, cell cycle, and DNA damage response. Indicative of their important regulatory functions, aberrant expression, and function of some lncRNAs have been observed in several types of cancers.

In some instances, the RNA molecule lacking a poly(A) tail includes small non-coding RNAs such as microRNAs (miRNAs), small interfering RNAs (siRNAs), Piwi-interacting RNAs (piRNAs), and small nucleolar RNAs (snoRNAs). In some instances, the RNA molecule lacking a poly(A) tail includes long intervening/intergenic noncoding RNAs (lincRNAs).

In some embodiments, examples of the RNA molecules lacking a poly(A) tail include, but are not limited to, ribosomal RNA (rRNA), mitochondrial RNA (mtRNA), transfer RNA (tRNA), microRNA (miRNA), and viral RNA. In some embodiments, the RNA molecules lacking a poly(A) tail can be a transcript (e.g., present in a tissue section).

In some embodiments, mRNA is not targeted for capture by randomer capture probes. In some embodiments, one or more randomer capture probes do not have a poly(T) sequence (e.g., also called a poly-thymine sequence, or a poly-thymidated sequence interchangeably) that can hybridize to the poly-A tail of eukaryotic mRNA. In yet another particular embodiment, the randomer capture probe targets and specifically hybridizes to one or more lncRNAs or microRNAs, for example.

In some embodiments, the one or more RNA molecules lacking a poly(A) tail is a single species of RNA. For example, in some embodiments, the one or more RNA molecule lacking a poly(A) tail are lncRNA molecules. In some embodiments, the one or more RNA molecule lacking a poly(A) tail are miRNA molecules. In some embodiments, the RNA molecule lacking a poly(A) tail can be a combination of two or more species of RNA. In some embodiments, the RNA molecule lacking a poly(A) tail is an RNA fragment of one of the RNA molecules lacking a poly(A) tail described herein. In some embodiments, the RNA molecule lacking a poly(A) tail is a full length RNA molecule of one of the RNA molecules lacking a poly(A) tail described herein.

### (b) Randomer Capture Probes

Disclosed herein are randomer capture probes that hybridize to RNA lacking poly(A) tails (e.g., lncRNAs, miRNAs). In some embodiments, the one or more randomer capture probes (interchangeably also called “randomer RNA capture probes,” “randomers,” “randomer probes,” “degenerate probes,” and the like throughout) is a DNA capture probe that is affixed to a substrate. It is appreciated that the randomer capture probe can be of a variety of lengths as disclosed herein. In specific instances, the randomer capture probe comprises a hexamer (e.g., a six-nucleotide sequence). In other specific instances, the randomer capture probe comprises a nonomer (e.g., a nine-nucleotide sequence). In some instances, the randomer capture probes include a random sequence of at least 6 to about 12 nucleotides in length (e.g., 6, 7, 8, 9, 10, 11, or 12).

In some instances, a single group of randomer capture probes (e.g., only nonomers; only hexamers) are used. In some instances, a combination of randomer capture probes are used. For instance, nonomers and hexamers can be used together at any ratio determined by a user (e.g. at an equal ratio; at a ratio in which there are more nonomers than hexamers, and vice versa). In some instances, the randomer capture probes are distributed onto a substrate, generating an array. Accordingly, in some instances, the randomer capture probes are distributed onto a substrate upon which a biological sample is placed. In some instances, both non-randomer capture probes (e.g., poly(T) capture probes, defined sequence capture probes) and randomer capture probes are distributed onto the substrate. In some embodiments, the capture probes and the randomer capture probes are distributed substantially evenly on the array, and/or wherein concentration of the capture probes and concentration of the randomer capture probes on the array is substantially the same. In some instances, the concentration of the capture probes on the array is higher than concentration of the randomer capture probes on the array. In some instances, the concentration of the capture probes one the array is lower than concentration of the randomer capture probes on the array.

In some instances, additional sequences are tagged onto the randomer capture probe at the 5′ end and/or the 3′ end of the randomer capture probe. For instance, a primer sequence (e.g., TAGTCGA (SEQ ID NO:1)) can be added to the 5′ end of the randomer capture probe sequence. In some instances, a spatial barcode is included in the randomer capture probe, thereby allowing for the spatial determination and correlation of the RNA molecule lacking a poly(A) tail to its location in the biological sample. Randomer capture probes of varying lengths can be optimized in order to find ideal melting temperatures (Tm) during hybridization of the randomer capture probe to an RNA molecule lacking a poly(A) tail (e.g., lncRNA). In some instances, the randomer capture probe also includes one or more functional domains, a unique molecular identifier, a cleavage domain, and combinations thereof.

In some instances, the randomer sequence (e.g., the hexamer or nonomer sequence) is located at the 3′ end of the randomer capture probe.

In some instances, the length of the randomer capture probe affixed to the substrate ranges from about 6 nucleotides to about 40 nucleotides (6, 7, 8, 9, 10, 11, 12, 13, 14, 15, 16, 20 17, 18, 19, 20, 21, 22, 23, 24, 25, 26, 27, 28, 29, 30, 31, 32, 33, 34, 35, 36, 37, 38, 39, or 40 nucleotides) in length.

In some embodiments, the randomer capture probe is a DNA probe. In some instances, the DNA probe includes a single-stranded DNA oligonucleotide having a sequence partially or completely complementary (and thus hybridizes) to an RNA molecule lacking a poly(A) tail (e.g., one or more lncRNA molecules). In some embodiments, the one or more randomer capture probes are at least 70%, at least 75%, at least 80%, at least 85%, at least 90%, at least 91%, at least 92%, at least 93%, at least 94%, at least 95%, at least 96%, at least 97%, at least 98%, or at least 99% complementary to one or more RNA molecules lacking a poly(A) tail (e.g., one or more lncRNA molecules). In some embodiments, the one or more randomer capture probes is 100% (i.e., completely) complementary to part of one or more RNA molecules lacking a poly(A) tail (e.g., one or more lncRNA molecules).

In some embodiments, any of the probes used herein can include elements described in Morlan et al., PLoS One. 2012;7(8):e42882, which is incorporated by reference in its entirety. In some embodiments, any of the probes used herein can include elements described in U.S. Appl. Publ. No. 2011/0111409, which is incorporated by reference in its entirety. In some embodiments, any of the probes used herein can include elements described in Adiconis et al., Nat Methods. 2013 Jul.; 10(7):623-9, which is incorporated by reference in its entirety.

The disclosure also provides methods of producing any one of the randomer capture probes described herein. In some instances, the randomer capture probes (e.g., a DNA probe) can be produced by techniques known in the art. For example, in some embodiments, the randomer capture probes (e.g., a DNA probe) are produced by chemical synthesis, by in vitro expression from recombinant nucleic acid molecules, or by in vivo expression from recombinant nucleic acid molecules. The randomer capture probe may also be produced by amplification e.g., RT-PCR, asymmetric PCR, or rolling circle amplification.

In some embodiments, the randomer probes once generated are affixed on a substrate to create the randomer capture probes by ligating the randomer probes to an existing capture probe already existing on a substrate (e.g., a capture probe that comprises a spatial barcode, functional sequences, and a sequence that is complementary to a splint oligonucleotide). For example, once the randomer probe is generated it can be ligated to the existing capture probe that has been previously affixed to a substrate using a splint oligonucleotide. Table 2 discloses a representative set of randomer capture probes that can serve as randomer capture domains and a splint oligonucleotide that can be used to attach the randomer capture domain to an existing capture probe on a substrate. Methods of attachment can be found in WO 2020/123305, incorporated herein by reference in its entirety.

In some embodiments, the randomer capture probe comprises at least one non-natural nucleic acid in its sequence. In some embodiments, the non-natural nucleic acid is a locked nucleic acid (LNA). In some embodiments, the randomer capture probe comprises one or more modifications to its structure.

Locked nucleic acids are a type of nucleic acid analog that contains a 2′-O, 4′-C methylene bridge, which increases the affinity for complementary RNA or DNA. In some instances, compared to naturally-occurring oligonucleotides, LNAs provide enhanced stability, increased melting temperature, and binding affinity. This bridge—locked in the 3′-endo conformation restricts the flexibility of the ribofuranose ring and locks the structure into a rigid bicyclic formation. LNAs are used to increase the sensitivity and specificity of molecular biology tools such as DNA microarrays and LNA-based oligonucleotides are being developed as antisense therapies. LNA can be incorporated into the randomer capture probe by standard phophoramidite chemistry.

In some embodiments, the LNA is a 2′-O,4′-C-methylene-a-1-ribofuranose (α-L-LNA) or a 2′-O,4′-C-methylene-β-d-ribofuranose (β-D-LNA). In some embodiments, the randomer capture probe includes all LNA residues, a LNA mixmer (any combination of LNA and DNA residues), a LNA gapmer (with a central DNA moiety flanked by LNA-modified 5′- or 3′-end), an LNA-modified LNAzyme, or any combinations thereof. Detailed descriptions of LNA can be founds, e.g., in Grunweiler and Roland, BioDrugs 21.4 (2007): 235-243, which is incorporated by reference in its entirety.

In some embodiments, the randomer capture probe comprises one or more (e.g., 1, 2, 3, 4, 5, 6, 7, 8, 9, 10, or more) modifications to its structure. For example, the modifications include one or more carbon moieties attached to the LNA. In some embodiments, the modifications include modified bases, e.g., 2′-O-methoxy-ethyl Bases (2′-MOE) (e.g., 2-MethoxyEthoxy A, 2-MethoxyEthoxy MeC, 2-MethoxyEthoxy G, and/or 2-MethoxyEthoxy T); 2′-O-Methyl RNA Bases (e.g., 2′-O-Methyl RNA Bases); Fluoro Bases (e.g., Fluoro C, Fluoro U, Fluoro A, and/or Fluoro G); 2-Aminopurine, 5-Bromo dU, deoxyUridine, 2,6-Diaminopurine (2-Amino-dA), Dideoxy-C, deoxyInosine, Hydroxymethyl dC, inverted dT, Iso-dG, Iso-dC, inverted Dideoxy-T, 3′-3′-inverted thymine, 5-Methyl dC, 5-Nitroindole, Super T (5-hydroxybutynl-2′-deoxyuridine), Super G (8-aza-7-deazaguanosine) and/or combinations thereof.

In some embodiments, the randomer capture probe comprises a mixture of DNA, RNA and/or LNA bases. Any one of the randomer capture probe nucleotides can be an LNA nucleotide. For instance, in a randomer that is a hexamer, any combination of the 1, 2, 3, 4, 5, or all 6 nucleotides can be LNA nucleotides. In instances with a nonomer, any combination of the 1, 2, 3, 4, 5, 6, 7, 8, or all 9 nucleotides can be LNA nucleotides.

In some instances, the LNA is complementary to a region of an RNA molecule lacking a poly(A) tail. In some instances, the LNA is about 60%, 65%, 70%, 75%, 80%, 85%, 90%, 91%, 92%, 93%, 94%, 95%, 96%, 97%, 98%, 99%, or 100% complementary to a region of an RNA molecule lacking a poly(A) tail. Thus, in some instances, the LNA specifically binds (e.g., hybridizes) to a complementary (e.g., 60%, 65%, 70%, 75%, 80%, 85%, 90%, 91%, 92%, 93%, 94%, 95%, 96%, 97%, 98%, 99%, or 100%) to the RNA molecule lacking a poly(A) tail.

In some embodiments, each LNA can increase melting temperature (Tm) of the randomer capture probe by at least 1° C., at least 2° C., at least 3° C., at least 4° C., or at least 5° C. as compared to a reference naturally-occurring oligonucleotide with the same sequence. In some embodiments, Tm of the randomer capture probe comprising one or more LNAs is about 10%, about 20%, about 30%, about 40%, about 50%, about 60%, about 70%, about 80%, about 90%, about 100%, or higher than Tm of a reference oligonucleotide with the same sequence except that the LNA is replaced by the corresponding oligonucleotide. In some embodiments, the higher Tm of the randomer capture probe comprising one or more LNAs leads to its increased binding affinity to the RNA molecule lacking a poly(A) tail. In some embodiments, the randomer capture probe comprising one or more LNAs has a similar Tm compared to a reference oligonucleotide sharing an identical sequence without LNAs, in which case the randomer capture probe is at least 1, 2, 3, 4, 5, 6, 7, 8, 9, 10, 11, 12, 13, 14, 15, 16, 17, 18, 19, 20 nucleotides shorter than the reference oligonucleotide. In some embodiments, the length of the randomer capture probe, and number and/or positions of incorporated LNAs are determined according to the randomer capture probe's Tm. In some embodiments, the Tm of the randomer capture probe is determined according to the highest temperature used during the spatial analysis workflow (e.g., extension or amplification).

In some embodiments, the one or more LNAs within the randomer capture probe can increase its binding affinity to the undesirable nucleic acid by at least 1 fold, at least 5 fold, at least 10 fold, at least 20 fold, at least 30 fold, at least 40 fold, at least 50 fold, at least 60 fold, at least 70 fold, at least 80 fold, at least 90 fold, at least 100 fold, or more as compared to the binding affinity of the same randomer capture probe except that the LNA is replaced by the corresponding DNA. In some embodiments, because of the increased binding affinity to the RNA molecule lacking a poly(A) tail, the randomer capture probe does not disassociate from the RNA molecule lacking a poly(A) tail during the extension (e.g., reverse transcription) and/or the amplification (e.g., PCR amplification) steps following the spatial analysis workflow.

In some embodiments, the methods of targeted RNA capture as disclosed herein include multiple randomer capture probes. In some embodiments, the randomer capture probes include sequences that are complementary or substantially complementary to one or more RNA molecules lacking a poly(A) tail. Methods provided herein may be applied to a single RNA molecule lacking a poly(A) tail or a plurality of RNA molecules lacking a poly(A) tail.

In some embodiments, the randomer capture probe is about 5, about 6, about 7, about 8, about 9, about 10, about 12, about 15, about 20, about 25, about 30, about 40, nucleotides in length.

In some embodiments, a single randomer capture probe spans the entire length of the RNA lacking a poly(A) tail (e.g., one or more lncRNA molecules). In some embodiments, the randomer capture probe has regions that are not complementary to RNA lacking a poly(A) tail, so long as such sequences do not substantially affect specific hybridization of the randomer capture probe to the target RNA (e.g., one or more lncRNA molecules).

### (c) Detection of Additional Analytes Using Depletion Probes or Templated Ligation Probes

In some instances, the methods and compositions relating to detection of an RNA molecule lacking a poly(A) sequence (e.g.,lncRNA or miRNA) can be combined with additional embodiments to detect and/or deplete other RNA molecules. For instance, in some embodiments, the methods include determining the location and/or abundance of one or more RNA molecules lacking a poly(A) sequence (e.g., lncRNA or miRNA) while depleting the concentration or abundance of one or more undesirable RNA molecules. As used herein, the term “undesirable RNA molecule”, or “undesirable RNA”, refers to an undesired RNA that is the target for depletion from the biological sample. In some embodiments, examples of the undesirable RNA include, but are not limited to, messenger RNA (mRNA), ribosomal RNA (rRNA), mitochondrial RNA (mtRNA), transfer RNA (tRNA), and viral RNA. In some embodiments, the undesirable RNA can be a transcript (e.g., present in a tissue section).

In some embodiments, the undesirable RNA molecule includes 5.8S ribosomal RNA (rRNA), 5S rRNA, transfer RNA (tRNA), a small nucleolar RNA (snoRNAs), Piwi-interacting RNA (piRNA), tRNA-derived small RNA (tsRNA), and small rDNA-derived RNA (srRNA), or mitochondrial RNA (mtRNA). In some embodiments, the undesirable RNA molecule includes an RNA molecule that is added (e.g., transfected) into a sample (e.g., a small interfering RNA (siRNA)). The undesirable RNA can be double-stranded RNA or single-stranded RNA. In embodiments where the undesirable RNA is double-stranded it is processed as a single-stranded RNA prior to depletion. In some embodiments, the undesirable RNA can be circular RNA. In some embodiments, the undesirable RNA can be a bacterial rRNA (e.g., 16s rRNA or 23s rRNA). In some embodiments, the undesirable RNA is from E. coli.

In some embodiments, the undesirable RNA molecule is rRNA. In some embodiments, the rRNA is eukaryotic rRNA. In some embodiments, the rRNA is cytoplasmic rRNA. In some embodiments, the rRNA is mitochondrial rRNA. Cytoplasmic rRNAs include, for example, 28S, 5.8S, 5S and 18S rRNAs. Mitochondrial rRNAs include, for example, 12S and 16S rRNAs. The rRNA may also be prokaryotic rRNA, which includes, for example, 5S, 16S, and 23S rRNA. The sequences for rRNAs are well known to those skilled in the art and can be readily found in sequence databases such as GenBank or may be found in the literature. For example, the sequence for the human 18S rRNA can be found in GenBank as Accession No. M10098 and the human 28S rRNA as Accession No. M11167.

In some embodiments, the undesirable RNA molecule is mitochondrial RNA. Mitochondrial RNAs include, for example, 12S rRNA (encoded by MT-RNR1), and 16S rRNA (encoded by MT-RNR2), RNAs encoding electron transport chain proteins (e.g., NADH dehydrogenase, coenzyme Q-cytochrome c reductase/cytochrome b, cytochrome c oxidase, ATP synthase, or humanin), and tRNAs (encoded by MT-TA, MT-TR, MT-TN, MT-TD, MT-TC, MT-TE, MT-TQ, MT-TG, MT-TH, MT-TI, MT-TL1, MT-TL2, MT-TK, MT-TM, MT-TF, MT-TP, MT-TS1, MT-TS2, MT-TT, MT-TW, MT-TY, or MT-TV).

In some embodiments, the undesirable RNA is transfer RNA (tRNA). In some embodiments, the undesirable RNA may be a particular mRNA. For example, it may be desirable to remove cellular transcripts that are usually present in abundance. Thus, the undesirable mRNA may include, but is not limited to, ACTB, GAPDH, and TUBB. Other sequences for tRNA and specific mRNA are well known to those skilled in the art and can be readily found in sequence databases such as GenBank or may be found in the literature.

In some embodiments, mRNA is not targeted for depletion by undesirable RNA probes. In yet another particular embodiment, the undesirable RNA depletion probe targets and specifically hybridizes to human 18S or human 28S rRNA. Examples of the sequence of undesirable RNA depletion probes targeting the full length sequence of human 18S and human 28S rRNA are illustrated in, e.g., US Appl. Publ. No. 2011/0111409 A1, which is incorporated herein by reference.

In some embodiments, the one or more undesirable RNA molecule is a single species of RNA. For example, in some embodiments, the one or more undesirable RNA molecule hybridizes only ribosomal RNA molecules. In some embodiments, the one or more undesirable RNA molecule hybridizes only mitochondrial RNA molecules. In some embodiments, the undesirable RNA molecule can be a combination of two or more species of RNA. In some embodiments, the undesirable RNA molecule is an RNA fragment of one of the undesirable RNA molecules described herein. In some embodiments, the undesirable RNA molecule is a full length RNA molecule of one of the undesirable RNA molecules described herein.

In additional embodiments, the detection methods herein can be combined with methods of detecting RNA by hybridizing analytes directly to one or more capture probes on an array, as described in WO 2020/176788 and U.S. Patent Application Publication No. 2020/0277663, each of which is incorporated by reference in its entirety. In another embodiment, the detection methods herein can be combined with methods of detecting RNA using spatial templated ligation. Methods of spatial templated ligation are described in U.S. Patent Application Publication No. 2021/0285046 and WO 2021/133849, each of which is incorporated by reference in its entirety.

As such, in the same spatial assay a sample can be depleted of undesirable RNA species while concurrently or sequentially capturing and detecting non-polyadenylated RNA species such and lncRNA and/or miRNA, further in combination with capturing and detecting mRNA targets of interest. By depleting undesirable RNA species, targets such as lncRNA and/or miRNA and/or low-abundant mRNA species can be detected to a higher degree compared to a sample where undesirable RNA species are not depleted.

### (d) Pre-Hybridization Methods

### (i) Imaging and Staining

Prior to the capture of RNA molecules lacking poly(A) tails by randomer capture probes on the substrate, in some instances, biological samples can be stained using a wide variety of stains and staining techniques. In some instances, the biological sample is a section on a slide (e.g., a 5 μm section, a 7 μm section, a 10 μm section, etc.). In some instances, the biological sample is dried after placement onto a glass slide. In some instances, the biological sample is dried at 42° C. In some instances, drying occurs for about 1 hour, about 2, hours, about 3 hours, or until the sections become transparent. In some instances, the biological sample can be dried overnight (e.g., in a desiccator at room temperature).

In some embodiments, a sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the methods disclosed herein include imaging the biological sample. In some instances, imaging the sample occurs prior to deaminating the biological sample. In some instances, the sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some instances, the stain is an H&E stain.

In some embodiments, while on the substrate, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes identifying analytes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

In some embodiments, biological samples can be destained. Methods of destaining or discoloring a biological sample are known in the art, and generally depend on the nature of the stain(s) applied to the sample. For example, H&E staining can be destained by washing the sample in HCl, or any other acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some embodiments, destaining can include 1, 2, 3, 4, 5, or more washes in an acid (e.g., HCl). In some embodiments, destaining can include adding HCl to a downstream solution (e.g., permeabilization solution). In some embodiments, destaining can include dissolving an enzyme used in the disclosed methods (e.g., pepsin) in an acid (e.g., HCl) solution. In some embodiments, after destaining hematoxylin with an acid, other reagents can be added to the destaining solution to raise the pH for use in other applications. For example, SDS can be added to an acid destaining solution in order to raise the pH as compared to the acid destaining solution alone. As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, immunofluorescence or immunohistochemistry protocols (direct and indirect staining techniques) can be performed as a part of, or in addition to, the exemplary spatial workflows presented herein. For example, tissue sections can be fixed according to methods described herein. The biological sample can be transferred to an array (e.g., capture probe array), wherein analytes (e.g., proteins) are probed using immunofluorescence protocols. For example, the sample can be rehydrated, blocked, and permeabilized (3X SSC, 2% BSA, 0.1% Triton X, 1 U/μl RNAse inhibitor for 10 minutes at 4° C.) before being stained with fluorescent primary antibodies (1:100 in 3XSSC, 2% BSA, 0.1% Triton X, 1 U/μl RNAse inhibitor for 30 minutes at 4° C.). The biological sample can be washed, coverslipped (in glycerol +1 U/μl RNAse inhibitor), imaged (e.g., using a confocal microscope or other apparatus capable of fluorescent detection), washed, and processed according to analyte capture or spatial workflows described herein.

In some instances, a glycerol solution and a cover slip can be added to the sample. In some instances, the glycerol solution can include a counterstain (e.g., DAPI).

As used herein, an antigen retrieval buffer can improve antibody capture in IF/IHC protocols. An exemplary protocol for antigen retrieval can be preheating the antigen retrieval buffer (e.g., to 95° C.), immersing the biological sample in the heated antigen retrieval buffer for a predetermined time, and then removing the biological sample from the antigen retrieval buffer and washing the biological sample.

In some embodiments, optimizing permeabilization can be useful for identifying intracellular analytes. Permeabilization optimization can include selection of permeabilization agents, concentration of permeabilization agents, and permeabilization duration. Tissue permeabilization is discussed elsewhere herein.

In some embodiments, blocking an array and/or a biological sample in preparation of labeling the biological sample decreases nonspecific binding of the antibodies to the array and/or biological sample (decreases background). Some embodiments provide for blocking buffers/blocking solutions that can be applied before and/or during application of the label, wherein the blocking buffer can include a blocking agent, and optionally a surfactant and/or a salt solution. In some embodiments, a blocking agent can be bovine serum albumin (BSA), serum, gelatin (e.g., fish gelatin), milk (e.g., non-fat dry milk), casein, polyethylene glycol (PEG), polyvinyl alcohol (PVA), or polyvinylpyrrolidone (PVP), biotin blocking reagent, a peroxidase blocking reagent, levamisole, Carnoy's solution, glycine, lysine, sodium borohydride, pontamine sky blue, Sudan Black, trypan blue, FITC blocking agent, and/or acetic acid. The blocking buffer/blocking solution can be applied to the array and/or biological sample prior to and/or during labeling (e.g., application of fluorophore-conjugated antibodies) to the biological sample.

### (ii) Preparation of a Sample for Capture of Target Analytes

In some instances, the biological sample is a fixed sample, for example the biological sample is a formalin fixed paraffin embedded or FFPE sample. When using FFPE samples, the sample should be deparaffinized and decrosslinked prior to the spatial workflow or order to make the target analytes accessible for capture and detection. Deparaffinization can be achieved using any method known in the art. For example, in some instances, the biological samples is treated with a series of washes that include xylene and various concentrations of ethanol. In some instances, methods of deparaffinization include treatment of xylene (e.g., three washes at 5 minutes each). In some instances, the methods further include treatment with ethanol (e.g., 100% ethanol, two washes 10 minutes each; 95% ethanol, two washes 10 minutes each; 70% ethanol, two washes 10 minutes each; 50% ethanol, two washes 10 minutes each). In some instances, after ethanol washes, the biological sample can be washed with deionized water (e.g., two washes for 5 minutes each). It is appreciated that one skilled in the art can adjust these methods to optimize deparaffinization.

In some instances, the biological sample is decrosslinked. In some instances, the biological sample is decrosslinked in a solution containing TE buffer (comprising Tris and EDTA). In some instances, the TE buffer is basic (e.g., at a pH of about 9). In some instances, decrosslinking occurs at about 50° C. to about 80° C. In some instances, decrosslinking occurs at about 70° C. In some instances, decrosslinking occurs for about 1 hour at 70° C. Just prior to decrosslinking, the biological sample can be treated with an acid (e.g., 0.1 M HCl for about 1 minute). After the decrosslinking step, the biological sample can be washed (e.g., with 1×PBST).

In some embodiments, a biological sample if a fresh frozen sample which does not require deparaffinization or decrosslinking.

In some instances, the methods of preparing a biological sample for randomer probe capture includes steps of equilibrating and blocking the biological sample. In some instances, equilibrating is performed using a pre-hybridization (pre-Hyb) buffer. In some instances, the pre-Hyb buffer is RNase-free. In some instances, the pre-Hyb buffer contains no bovine serum albumin (BSA), solutions like Denhardt's, or other potentially nuclease-contaminated biological materials.

In some instances, the equilibrating step is performed multiple times (e.g., 2 times at 5 minutes each; 3 times at 5 minutes each). In some instances, the biological sample is blocked with a blocking buffer. In some instances, the blocking buffer includes a carrier such as tRNA, for example yeast tRNA such as from brewer's yeast (e.g., at a final concentration of 10-20 μg/mL). In some instances, blocking can be performed for 5, 10, 15, 20, 25, or 30 minutes.

Any of the foregoing steps can be optimized for performance. For example, one can vary the temperature. In some instances, the pre-hybridization methods are performed at room temperature. In some instances, the pre-hybridization methods are performed at 4° C. (in some instances, varying the timeframes provided herein).

### (g) Permeabilization and Releasing RNA Molecules for Capture

In some embodiments, prior to allowing the RNA molecules lacking poly(A) sequences to be captured by the randomer capture probes on the array, the methods provided herein include a permeabilizing step. In some embodiments, permeabilization occurs using a protease. In some embodiments, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin. In some embodiments, the biological sample is permeabilized contemporaneously with or prior to contacting the biological sample with the substrate comprising randomer capture probes. In some embodiments, the biological sample is permeabilized after the biological sample is contacted with the substrate comprising randomer capture probes.

In some embodiments, methods provided herein include permeabilization of the biological sample such that the randomer capture probes can more easily bind to the RNA lacking a poly(A) tail (i.e., compared to no permeabilization). In some embodiments, reverse transcription (RT) reagents can be added to permeabilized biological samples. Incubation with the RT reagents can produce spatially-barcoded full-length cDNA from the captured analytes (e.g., RNA lacking a poly(A) tail, polyadenylated mRNA, etc.). Second strand reagents (e.g., second strand primers, enzymes) can be added to the biological sample on the slide to initiate second strand synthesis.

In some instances, the permeabilization step includes application of a permeabilization buffer to the biological sample. In some instances, the permeabilization buffer includes a buffer (e.g., Tris pH 7.5), MgCl₂, sarkosyl detergent (e.g., sodium lauroyl sarcosinate), enzyme (e.g., proteinase K), and nuclease free water. In some instances, the permeabilization step is performed at 37° C. In some instances, the permeabilization step is performed for about 20 minutes to 2 hours (e.g., about 20 minutes, about 30 minutes, about 40 minutes, about 50 minutes, about 1 hour, about 1.5 hours, or about 2 hours). In some instances, the releasing step is performed for about 40 minutes.

### (h) RNA Capture Using Arrays having Capture Probes having Poly-Thymine Sequences and/or Randomer Capture Probes

In some embodiments, analytes are released from the biological sample and are captured on an array comprising capture probes (e.g., poly(T)-containing capture probes and/or randomer capture probes). In some instances, one or more randomer capture probes hybridize to an RNA lacking a poly(A) tail (e.g., an lncRNA molecule). In some embodiments, one or more randomer capture domains of capture probes hybridize to the complete sequence of the RNA molecule lacking a poly(A) tail. Hybridization can occur with an analyte having a sequence that is 100% complementary to the randomer capture domain of the capture probe. In some embodiments, hybridization can occur with an analyte having a sequence that is at least (e.g., at least about) 80%, at least (e.g., at least about) 85%, at least (e.g., at least about) 90%, at least (e.g., at least about) 95%, at least (e.g., at least about) 96%, at least (e.g., at least about) 97%, at least (e.g., at least about) 98%, or at least (e.g., at least about) 99% complementary to the randomer capture domain of the capture probes.

In some embodiments, the randomer capture domain of the capture probe may be complementary to all or part of an RNA molecule lacking a poly(A) tail (e.g., lncRNA). For example, there may be at least 1, 2, 3, 4, 5, 6, 7, 8, 9, 10, or more randomer capture domains that specifically hybridize to an RNA molecule lacking a poly(A) tail. In some embodiments, the RNA molecule lacking a poly(A) tail has a tertiary structure and the randomer capture domain of a capture probe can be complementary to an exposed portion of the RNA molecule lacking a poly(A) tail. In some embodiments, one or more randomer capture domains can hybridize to the RNA molecule lacking a poly(A) tail such that at least about 1 to about 40 nucleotides of the RNA molecule lacking a poly(A) tail is hybridized to the randomer capture domain of the capture probe. After capture, and as described below, the randomer capture probe can be extended and further analyzed (e.g., by amplification and sequencing).

In some instances, the randomer capture probe is used concurrently with capture of additional spatial analysis targets, including spatial templated ligation, detection of RNA molecules having poly(A) sequences, and detection of proteins. Methods of detecting RNA molecules having poly(A) sequences and protein have been described previously in WO 2020/176788 and U.S. Patent Application Publication No. 2020/0277663, each of which is incorporated by reference in its entirety. Methods of spatial templated ligation are described in U.S. Patent Application Publication No. 2021/0285046 and WO 2021/133849, each of which is incorporated by reference in its entirety.

### (i) Biological Samples

Methods disclosed herein can be performed on any type of sample. In some embodiments, the sample is a fresh tissue. In some embodiments, the sample is a frozen sample. In some embodiments, the sample was previously frozen. In some embodiments, the sample is a fixed sample. In some embodiments, the sample is a formalin-fixed, paraffin embedded (FFPE) sample. In some instances, the biological sample is placed on substrate prior to contact of the biological sample with one or more randomer capture probes.

Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy. In some instances, the biological sample can include one or more diseased cells. A diseased cell can have altered metabolic properties, gene expression, protein expression, and/or morphologic features. Examples of diseases include inflammatory disorders, metabolic disorders, nervous system disorders, and cancer. In some instances, the biological sample includes cancer or tumor cells. Cancer cells can be derived from solid tumors, hematological malignancies, cell lines, or obtained as circulating tumor cells. In some instances, the biological sample is a heterogenous sample. In some instances, the biological sample is a heterogenous sample that includes tumor or cancer cells and/or stromal cells.

In some instances, the cancer is breast cancer. In some instances, the cancer is colorectal cancer. In some instances, the cancer is ovarian cancer. In certain embodiments, the cancer is squamous cell cancer, small-cell lung cancer, non-small cell lung cancer, gastrointestinal cancer, Hodgkin's or non-Hodgkin's lymphoma, pancreatic cancer, glioblastoma, glioma, cervical cancer, ovarian cancer, liver cancer, bladder cancer, breast cancer, colon cancer, colorectal cancer, endometrial carcinoma, myeloma, salivary gland carcinoma, kidney cancer, basal cell carcinoma, melanoma, prostate cancer, vulval cancer, thyroid cancer, testicular cancer, esophageal cancer, or a type of head or neck cancer. In certain embodiments, the cancer treated is desmoplastic melanoma, inflammatory breast cancer, thymoma, rectal cancer, anal cancer, or surgically treatable or non-surgically treatable brain stem glioma. In some embodiments, the subject is a human.

FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some instances, FFPE samples are stained (e.g., using H&E). The methods disclosed herein are compatible with H&E will allow for morphological context overlaid with transcriptomic analysis. However, depending on the need some samples may be stained with only a nuclear stain, such as staining a sample with only hematoxylin and not eosin, when location of a cell nucleus is needed.

In some embodiments, a biological sample (e.g., tissue section) can be fixed with methanol, stained with hematoxylin and eosin, and imaged. In some embodiments, fixing, staining, and imaging occurs before one or more probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

In some embodiments, the FFPE sample is deparaffinized, permeabilized, equilibrated, and blocked before RTL or capture of RNA lacking a poly(A) tail. In some embodiments, deparaffinization using xylenes. In some embodiments, deparaffinization includes multiple washes with xylenes. In some embodiments, deparaffinization includes multiple washes with xylenes followed by removal of xylenes using multiple rounds of graded alcohol followed by washing the sample with water. In some aspects, the water is deionized water. In some embodiments, equilibrating and blocking includes incubating the sample in a pre-Hyb buffer. In some embodiments, the pre-Hyb buffer includes yeast tRNA. In some embodiments, permeabilizing a sample includes washing the sample with a phosphate buffer. In some embodiments, the buffer is PBS. In some embodiments, the buffer is PBST.

### (j) Determining the Sequence of the Randomer Probes or Complements Thereof

After an analyte (e.g., mRNA molecule having or lacking a poly(A) sequence) from the sample has hybridized or otherwise been associated with a capture probe according to any of the methods described above in connection with the general spatial cell-based analytical methodology, the barcoded constructs that result from hybridization/association are analyzed.

In some embodiments, after contacting a biological sample with a substrate that includes capture probes, a removal step can optionally be performed to remove all or a portion of the biological sample from the substrate. Sample removal occurs after the analyte and/or ligation product is captured on a capture probe of a substrate. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

In some embodiments, a biological sample is not removed from the substrate. For example, the biological sample is not removed from the substrate prior to releasing a capture probe (e.g., a capture probe bound to an analyte) from the substrate. In some embodiments, at least a portion of the biological sample is not removed from the substrate. For example, a portion of the biological sample can remain on the substrate prior to releasing a capture probe (e.g., a capture prove bound to an analyte) from the substrate and/or analyzing an analyte bound to a capture probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of an analyte bound to a capture probe from the substrate.

In some embodiments, the method further includes subjecting a region of interest in the biological sample to spatial transcriptomic analysis. In some embodiments, one or more of the capture probes includes a capture domain (e.g., a randomer capture domain, a poly(T) capture domain, a fixed and known sequence capture domain). In some embodiments, one or more of the capture probes comprises a unique molecular identifier (UMI). In some embodiments, one or more of the capture probes comprises a cleavage domain. In some embodiments, the cleavage domain comprises a sequence recognized and cleaved by uracil-DNA glycosylase, apurinic/apyrimidinic (AP) endonuclease (APE1), uracil-specific excision reagent (USER), and/or an endonuclease VIII. In some embodiments, one or more capture probes do not comprise a cleavage domain and is not cleaved from the array.

In some embodiments, after performing RNA capture methods disclosed herein, methods for spatially detecting an analyte (e.g., detecting the location of an analyte, e.g., a biological analyte) from a biological sample (e.g., present in a biological sample) are performed. In some instances, the methods include determining (i) all or part of the sequence of the RNA molecule lacking the poly(A) sequence bound to the randomer capture probe to determine and correlate the abundance and the spatial location of the RNA molecule lacking the poly(A) sequence in the biological sample.

Methods of extending the capture probe (e.g., randomer capture probe or poly(T)-containing capture probe) have been disclosed previously in WO 2020/176788 and/or U.S. Patent Application Publication No. 2020/0277663, each of which is incorporated by reference.

In some instances, in addition to determining and correlating the abundance and the location of the RNA molecule lacking the poly(A) sequence in the biological sample, a capture probe (e.g., a randomer capture probe, a poly(T) capture probe) can be extended (an “extended capture probe,” e.g., as described herein). For example, extending a capture probe can include generating cDNA from a captured (hybridized) RNA molecule. This process involves synthesis of a complementary strand of the hybridized nucleic acid, e.g., generating cDNA based on the captured RNA template (the RNA hybridized to the capture domain of the capture probe). Thus, in an initial step of extending a capture probe, e.g., the cDNA generation, the captured (hybridized) nucleic acid, e.g., RNA, acts as a template for the extension, e.g., reverse transcription, step.

In some instances, in addition to determining the abundance and/or the location of the RNA molecule lacking the poly(A) sequence in the biological sample, the capture probe is extended using reverse transcription. For example, reverse transcription includes synthesizing cDNA (complementary or copy DNA) from RNA using a reverse transcriptase. In some embodiments, reverse transcription is performed while the tissue is still in place, generating an analyte library, where the analyte library includes the spatial barcodes from the adjacent capture probes. In some embodiments, the capture probe is extended using one or more DNA polymerases.

In some instances, in addition to determining the abundance and/or the location of the RNA molecule lacking the poly(A) sequence in the biological sample, a capture domain of a capture probe includes a primer for producing the complementary strand of the analyte hybridized to the capture probe, e.g., a primer for DNA polymerase and/or reverse transcription. The nucleic acid, e.g., DNA and/or cDNA, molecules generated by the extension reaction incorporate the sequence of the capture probe. The extension of the capture probe, e.g., a DNA polymerase and/or reverse transcription reaction, can be performed using a variety of suitable enzymes and protocols.

In some instances, in addition to determining the abundance and/or the location of the RNA molecule lacking the poly(A) sequence in the biological sample, a full-length DNA (e.g., cDNA) molecule is generated. In some embodiments, a “full-length” DNA molecule refers to the whole of the captured nucleic acid molecule. However, if a nucleic acid (e.g., RNA) was partially degraded in the tissue sample, then the captured nucleic acid molecules will not be the same length as the initial RNA in the tissue sample. In some embodiments, the 3′ end of the extended probes, e.g., first strand cDNA molecules, is modified. For example, a linker or adaptor can be ligated to the 3′ end of the extended probes. This can be achieved using single stranded ligation enzymes such as T4 RNA ligase or Circligase™ (available from Lucigen, Middleton, Wis.). In some embodiments, template switching oligonucleotides are used to extend cDNA in order to generate a full-length cDNA (or as close to a full-length cDNA as possible). In some embodiments, a second strand synthesis helper probe (a partially double stranded DNA molecule capable of hybridizing to the 3′ end of the extended capture probe), can be ligated to the 3′ end of the extended probe, e.g., first strand cDNA, molecule using a double stranded ligation enzyme such as T4 DNA ligase. Other enzymes appropriate for the ligation step are known in the art and include, e.g., Tth DNA ligase, Taq DNA ligase, Thermococcus sp. (strain 9° N) DNA ligase (9° N™ DNA ligase, New England Biolabs), Ampligase™(available from Lucigen, Middleton, Wis.), and SplintR (available from New England Biolabs, Ipswich, Mass.). In some embodiments, a polynucleotide tail, e.g., a poly(A) tail, is incorporated at the 3′ end of the extended probe molecules. In some embodiments, the polynucleotide tail is incorporated using a terminal transferase active enzyme.

In some instances, in addition to determining the abundance and the location of the RNA molecule lacking the poly(A) sequence in the biological sample, double-stranded extended capture probes are treated to remove any unextended capture probes prior to amplification and/or analysis, e.g., sequence analysis. This can be achieved by a variety of methods, e.g., using an enzyme to degrade the unextended probes, such as an exonuclease enzyme, or purification columns.

In some embodiments, extended capture probes are amplified to yield quantities that are sufficient for analysis, e.g., via DNA sequencing. In some embodiments, the first strand of the extended capture probes (e.g., DNA and/or cDNA molecules) acts as a template for the amplification reaction (e.g., a polymerase chain reaction).

In some embodiments, the amplification reaction incorporates an affinity group onto the extended capture probe (e.g., RNA-cDNA hybrid) using a primer including the affinity group. In some embodiments, the primer includes an affinity group and the extended capture probes includes the affinity group. The affinity group can correspond to any of the affinity groups described previously.

In some embodiments, the extended capture probes including the affinity group can be coupled to a substrate specific for the affinity group. In some embodiments, the substrate can include an antibody or antibody fragment. In some embodiments, the substrate includes avidin or streptavidin and the affinity group includes biotin. In some embodiments, the substrate includes maltose and the affinity group includes maltose-binding protein. In some embodiments, the substrate includes maltose-binding protein and the affinity group includes maltose. In some embodiments, amplifying the extended capture probes can function to release the extended probes from the surface of the substrate, insofar as copies of the extended probes are not immobilized on the substrate.

In some embodiments, the extended capture probe or complement or amplicon thereof is released. The step of releasing the extended capture probe or complement or amplicon thereof from the surface of the substrate can be achieved in a number of ways. In some embodiments, an extended capture probe, or a complement thereof, is released from the array by nucleic acid cleavage and/or by denaturation (e.g., by heating to denature a double-stranded molecule).

In some embodiments, the extended capture probe or complement or amplicon thereof is released from the surface of the substrate (e.g., array) by physical means. For example, where the extended capture probe is indirectly immobilized on the array substrate, e.g., via hybridization to a surface probe, it can be sufficient to disrupt the interaction between the extended capture probe and the surface probe. Methods for disrupting the interaction between nucleic acid molecules include denaturing double stranded nucleic acid molecules are known in the art. A straightforward method for releasing the DNA molecules (i.e., of stripping the array of extended probes) is to use a solution that interferes with the hydrogen bonds of the double stranded molecules. In some embodiments, the extended capture probe is released by an applying heated solution, such as water or buffer, of at least 85° C., e.g., at least 90, 91, 92, 93, 94, 95, 96, 97, 98, or 99° C. In some embodiments, a solution including salts, surfactants, etc. that can further destabilize the interaction between the nucleic acid molecules is added to release the extended capture probe from the substrate.

In some embodiments, where the extended capture probe includes a cleavage domain, the extended capture probe is released from the surface of the substrate by cleavage. For example, the cleavage domain of the extended capture probe can be cleaved by any of the methods described herein. In some embodiments, the extended capture probe is released from the surface of the substrate, e.g., via cleavage of a cleavage domain in the extended capture probe, prior to the step of amplifying the extended capture probe.

In some instances, the analyte and capture probe can be amplified or copied, creating a plurality of cDNA molecules. In some instances, the ligated probe and capture probe can be amplified or copied, creating a plurality of cDNA molecules. In some embodiments, cDNA can be denatured from the capture probe template and transferred (e.g., to a clean tube) for amplification, and/or library construction. The spatially-barcoded cDNA can be amplified via PCR prior to library construction. The cDNA can then be enzymatically fragmented and size-selected in order to optimize for cDNA amplicon size. P5 and P7 sequences directed to capturing the amplicons on a sequencing flowcell (Illumina sequencing instruments) can be appended to the amplicons, i7, and i5 can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites. The additional sequences are directed toward Illumina sequencing instruments or sequencing instruments that utilize those sequences; however a skilled artisan will understand that additional or alternative sequences used by other sequencing instruments or technologies are also equally applicable for use in the aforementioned methods.

In some embodiments, where a sample is barcoded directly via hybridization with capture probes or analyte capture agents hybridized, bound, or associated with either the cell surface, or introduced into the cell, as described above, sequencing can be performed on the intact sample.

After generation of the extended capture probe, the single strand of the extended capture probe is denatured from the strand that is attached to the substrate, thereby freeing a single strand of the extended capture probe for further analysis. In some instances, one strand of the extended capture probe remains on the substrate and can be used to generate another round (or multiple rounds) of extended capture probes.

Once the extended capture probe is released from the substrate, P5 and P7 sequences directed to capturing the amplicons on a sequencing flowcell (Illumina sequencing instruments) can be appended to the extended capture probes. After a round of sample index PCR, the extended capture probe can be sequenced.

### (k) Sequencing

A wide variety of different sequencing methods can be used herein. In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

### (1) Kits

In some embodiments, also provided herein are kits that include one or more reagents to detect one or more analytes described herein. In some instances, the kit includes a substrate comprising a plurality of capture probes comprising a spatial barcode and the capture domain comprising randomer sequences or homopolymeric sequences or defined sequences as described herein. In some instances, the kit includes a plurality of probes not affixed to a substrate surface (e.g., sets of RTL probes; RD probes).

A non-limiting example of a kit used to perform any of the methods described herein includes: (a) a substrate comprising a plurality of capture probes comprising a spatial barcode and a capture domain; (b) wherein a capture domain is a randomer capture domain which is substantially complementary to a sequence of an RNA molecule lacking a poly(A) tail (e.g., lncRNA) in the biological sample; and (c) instructions for performing any of the methods described herein.

A further non-limiting example of a kit used to perform any of the methods described herein includes: (a) a substrate comprising a plurality of capture probes comprising a spatial barcode and a capture domain, wherein a capture domain comprises either (i) a randomer capture domain, (ii) a poly(T) capture domain, or (iii) a defined sequence capture domain; (b) a system comprising: a first RTL probe, a second RTL probe, and a plurality of randomer capture probes, wherein the first RTL probe and the second RTL probe are substantially complementary to adjacent sequences of the analyte, wherein the second RTL probe comprises a capture probe binding domain that is capable of binding to a capture domain of a capture probe, and wherein a randomer domain of a capture probe of the plurality of capture probes is substantially complementary to a sequence of an RNA molecule lacking a poly(A) tail in the biological sample; and (c) instructions for performing any of the methods described herein.

### EXAMPLES

### Example 1. Capture of RNA Molecules Lacking a Poly(A) Tail

In this example, RNA molecules lacking a poly(A) tail such as lncRNA or miRNAs are captured on an array comprising randomer capture probes. A biological tissue section is placed on the array, wherein the capture probes on the array include either single-stranded nonomer and/or single-stranded hexamer capture domain sequences. At the same time, in some conditions, free-floating rRNA depletion probes that target ribosomal RNA and/or mitochondrial RNA are added to the biological sample and hybridized to ribosomal RNA and/or mitochondrial RNA. The biological sample is permeabilized and RNA molecules lacking a poly(A) tail are captured on the array by hybridization to the randomer capture domains of the capture probes. After capture, the randomer capture probes are extended using the RNA molecule lacking a poly(A) tail as a template. The extended randomer capture probe are amplified, purified, and sequenced.

### Example 2. Capture of Both RNA Molecules Lacking a Poly(A) Tail and Templated Ligation Molecules

In this example, RNA molecules lacking a poly(A) tail such as lncRNA or miRNAs are captured on an array comprising both randomer capture probes and capture probes comprising poly-thymine sequences. Concurrently with capture of the RNA molecules lacking a poly(A) tail, additional target analytes are detected using RTL probes. A biological tissue section is placed on an array. RTL probes (i.e., LHS and RHS probes) are applied to the sample (optionally simultaneously with the rRNA depletion probes described in Example 1). The RTL probes hybridize to their targets and a ligation step ligates the RTL probes together. RNase H digestion of the RNA of the DNA:RNA formed hybrids (after RTL probe hybridization), thereby digesting the rRNA and depleting those molecules (if rRNA depletion probes are present) while at the same time releasing the RTL ligation product. The sample is permeabilized, allowing both the RTL ligation products and the RNA molecules lacking poly(A) tails (e.g., lncRNA or miRNA) to hybridize to the poly-thymine capture probes or randomer capture probes, respectively, on the array. After hybridization, the 3′ end of the poly-thymine capture probe or randomer capture probe is extended using the RTL ligation product or the RNA molecules lacking poly(A) tails, respectively, as a template. The extended capture probes can be amplified and collected for downstream library preparation and subsequent spatial expression analysis.

### Example 3. Detection of RNA Molecules Lacking Poly(A) Tails

Detection of RNA molecules lacking poly(A) tails can be combined with other embodiments provided herein. For instances, the methods of detecting RNA molecules lacking poly(A) tails using randomer capture probes can be combined in the settings of undesirable RNA depletion and indiscriminate capture of poly(A) containing molecules using capture probes having poly(T) sequences. Here, randomer probes can specifically hybridize to RNA molecules lacking a poly(A) tail. In some instances, ribosomal depletion, or RD, probes are used. Then, the tissue sample can be permeabilized by any permeabilization methods as described herein in order to capture RNA molecules lacking poly(A) tails, other RNA molecules using poly(T) containing mRNA, or both.

For spatial transcriptomic analysis in combination with rRNA depletion experiments, in the reverse transcription (RT) step, H₂O was replaced with an equivalent volume of the pooled depletion probes (2 uM) in IDTE buffer (10 mM Tris, 0.1 mM EDTA, pH 7.5-8.0). The final concentration of each rRNA depletion probe in the RT reaction mixture was about 1 μm.

In the following experiment, two mouse brain samples were sectioned and analyzed for gene expression. As shown in Table 1, there were eight different experimental conditions: an array including poly(T) capture domains only, randomer capture domains (either hexamer or nonomer), or a combination thereof, with or without the addition of rRNA depletion probes (RD) during the spatial workflow.

TABLE 1
Probe Groups

| Group | Probes |
|---|---|
| A1 | Poly(T) Capture Probes |
| A2 | Poly(T) Capture Probes + Ribosomal Depletion (RD) Probes |
| B1 | Nonomer Randomer Capture Probes (“Nonomer”) |
| B2 | Nonomer Capture Probes + RD Probes |
| C1 | Hexamer Randomer Capture Probes (“Hexamer”) |
| C2 | Hexamer Randomer Capture Probes + RD Probes |
| D1 | Nonomer Random Capture Probes + Poly(T) Capture Probes |
| D2 | Nonomer Random Capture Probes+ Poly(T) Capture Probes + |
| | RD |

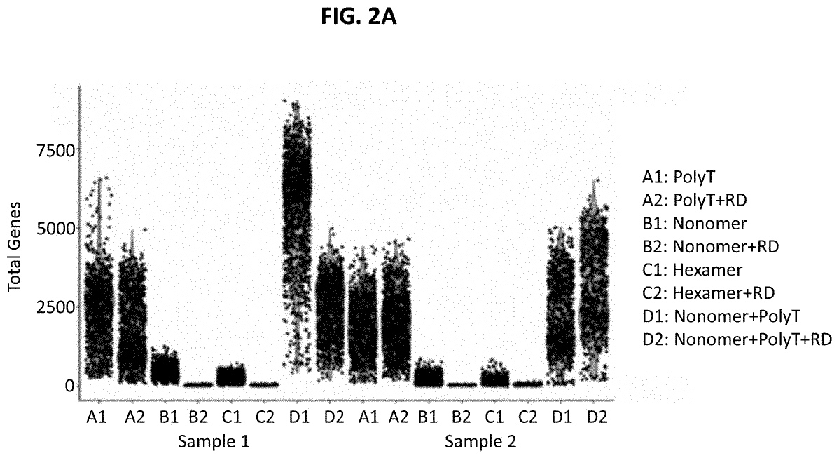

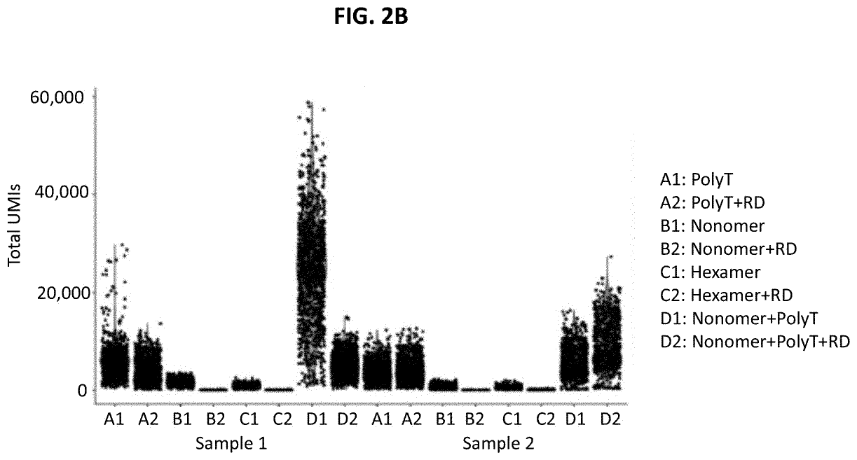

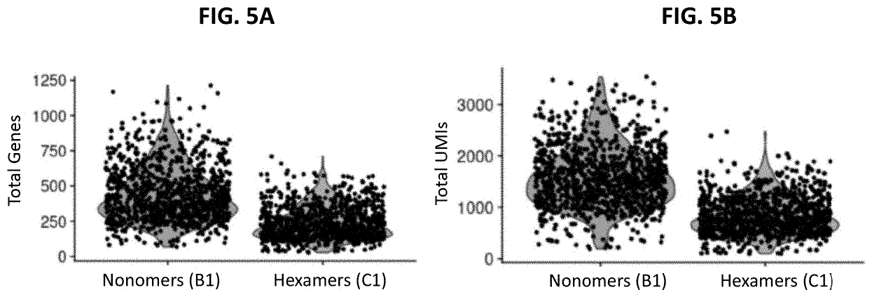

The methods described in Examples 1 and 2 were performed for each group, depending on the array (having only poly(T) capture probes, only randomer capture probes, or both) and whether depletion probes (RD probes) were used. Effects of global depletion of gene expression were assessed. As shown in FIG. 2A, the total gene number was reduced in Groups B1, B2, C1, and C2. Consistent with these observations, the total unique molecular identifiers (UMIs) were reduced in Groups B1, B2, C1, and C2. See FIG. 2B. Further, there was a decrease in capture of genes and UMIs when RD probes were used with randomers, demonstrating that (1) the randomer probes target RNA molecules lacking poly(A) sequences and (2) the RD probes compete for this group of RNA molecules. Use of nonomers alone appear to result in slightly more total genes and more UMIs compared to use of hexamers. FIGS. 5A and 5B. These data suggest that randomers (e.g., nonomers or hexamers)—either alone or in combination with ribosomal depletion (RD) probes—globally affect total gene expression and global capture of analytes.

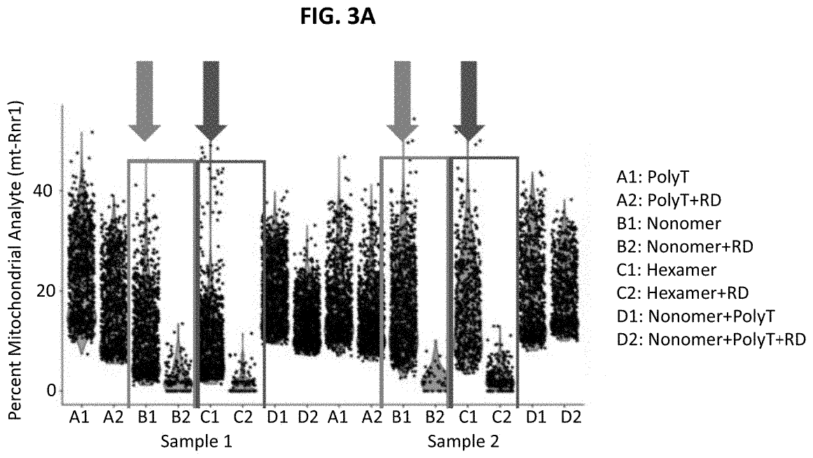

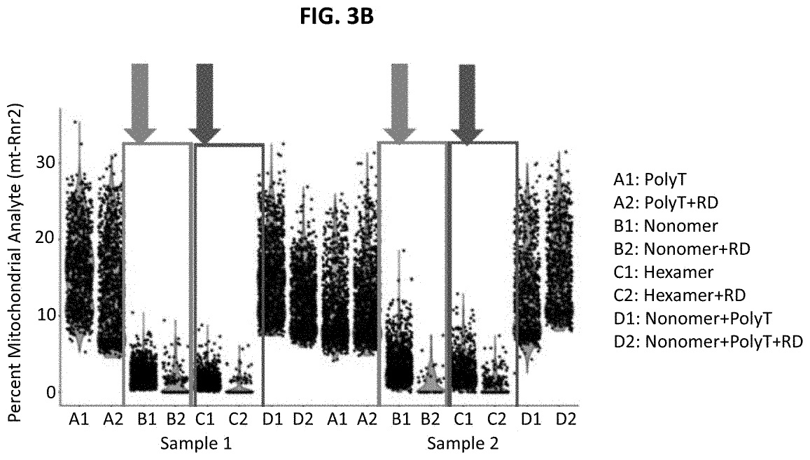

RD probes have been shown to target ribosomal and mitochondrial analytes. Two well-expressed mitochondrial analytes, mt-Rnr1 and mt-Rnr2, are targeted at least in part by RD probes. Thus, mt-Rnrl and mt-Rnr2, each of which are highly expressed in mouse brain, were separated from analyte analysis to determine specificity of randomer capture in conjunction with RD probe hybridization. Indeed, filtering mt-Rnr1 and mt-Rnr2 from the dataset reveals that Groups B2 and C2 (each of which comprise a randomer and RDs) appear to specifically target mt-Rnr1 and mt-Rnr2. See FIGS. 3A and 3B. These data suggest that the experimental RDs targeted mt-Rnr1 and mt-Rnr2, but that randomer capture of RNA molecules lacking a poly(A) tail does not affect mt-Rnr1 and mt-Rnr2 detection.

Individual gene targets were analyzed to determine whether the randomer capture domains captured any specific individual analytes. Nonomers and hexamers, Groups B1 and C1, respectively, appear to have an effect on the expression of proenkephalin (Penk), cholecystokinin (Cck), and potassium channel tetramerization domain containing 12 (Kctd12) in mouse brain. On the other hand, Groups B2 (nonomer+RD) and C2 (hexamer+RD) downregulate expression of mt-Rnr1 and mt-Rnr2 in mouse brain tissue. These data provide proof of principle that randomers and RDs can target and/or deplete specific targeted non-poly(A) analytes.

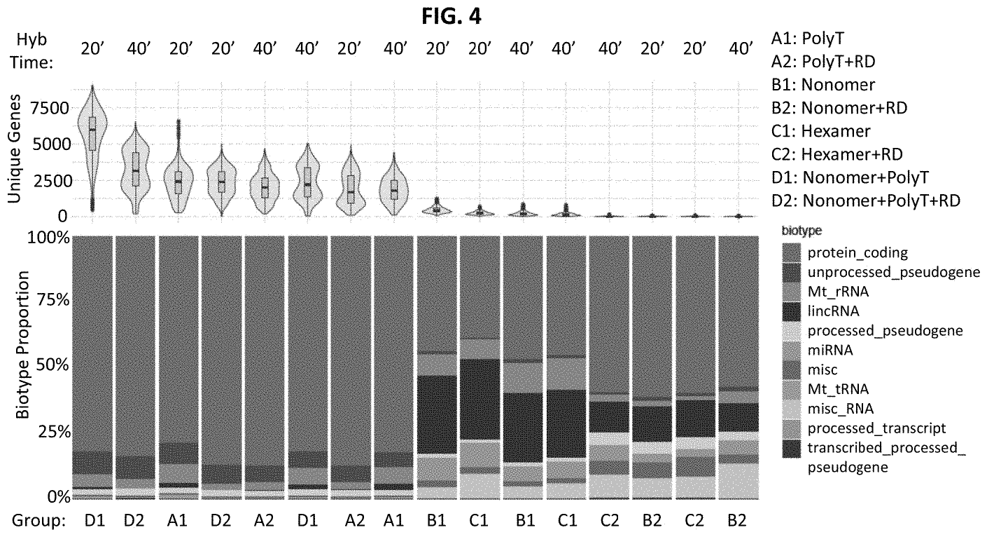

The type of analyte to which each condition in Table 1 hybridized was investigated. As shown in FIG. 4, while Groups A1, A2, D1, and D2 each appear to hybridize predominantly to protein coding analytes, regardless of hybridization time (e.g., either 20 minutes or 40 minutes), there was an appreciable increase in long noncoding RNA detection in Groups B1, B2, C1, and C2, regardless of hybridization time. These data suggest that randomer capture probes—either alone or with RD probes—target a distinct proportion of types of analytes that includes lncRNAs compared to groups comprising capture probes with a poly(T) sequence.

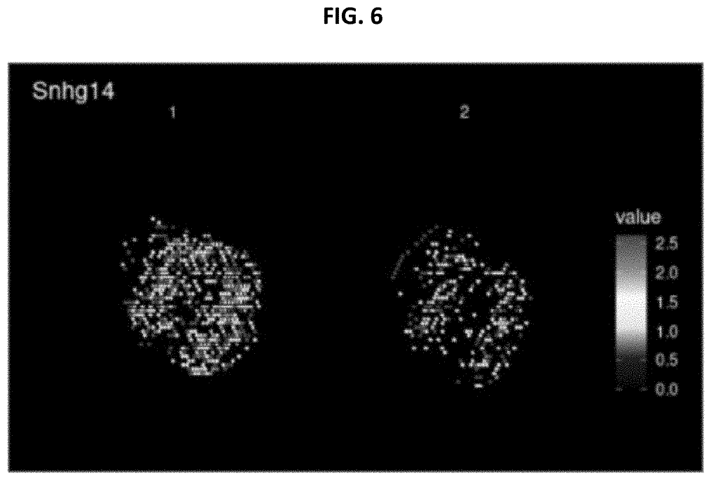

In samples wherein capture was performed only with nonomer capture probes or only with hexamer capture probes (e.g., B1 and C1, respectively) on an array, three predominant Seurat clusters were identified. At the same time, it was investigated whether nonomer capture probes and/or hexamer capture probes would organize with the clusters. Differential expression analysis in each Suerat cluster was determined, and a heat map of gene expression was generated. Notably, one of the most differentially expressed genes in each cluster was small nucleolar RNA host gene 14 (Snhg14). Snhg14 appears to be highly expressed with cluster 1 and less expressed in clusters 0 and 2. The spatial expression pattern of Snhg14 was generated, demonstrating differential expression in each sample. See FIG. 6. These data demonstrate the proof-of-concept ability to determine differentially-expressed analytes using randomer capture probes.

### Example 4. RNA Capture Using Locked Nucleic Acids (LNAs) Targeting RNA Molecules Lacking Poly(A) Tails

Randomer capture probes comprising non-natural nucleotides such as locked nucleic acids (LNAs) are an attractive modification to randomer capture probe synthesis because LNAs have increased stability compared to natural nucleic acids. To test whether using randomer capture probes with LNA nucleotides would increase overall capture efficiency, a series of randomer capture probes were designed as shown in Table 2, and used to create capture probes on spatial arrays using the splint oligonucleotide sequence listed. Methods of Examples 1 and/or 2 were performed to examine the spatial location and abundance of the captured analytes.

TABLE 2
LNA Randomer Capture Probes

| Concentration | 5′ modification | Name of probe | Sequence 5′-3′ |
|---|---|---|---|
| 1 uM | 5Phos | Nonomer-random | TAGTCGANNNNNNNNN |
| 5 uM | n/a | Splint_oligo | TCGACTACGTAGCG |
| 1 uM | 5Phos | 9N-v1_Tm48.6_LNA | TAGTCGA+N+N+N+N+N+NNNN |
| 1 uM | 5Phos | 9N-v2_Tm46.5_LNA | TAGTCGA+N+NN+NN+N+N+N+N |
| 1 uM | 5Phos | 9N-v3_Tm36.5_LNA | TAGTCGA+NNNN+NN+N+N+N |
| 1 uM | 5Phos | 9N-v4_Tm37.4_LNA | TAGTCGA+N+N+NN+NNNNN |

“+N”: LNA nucleotide

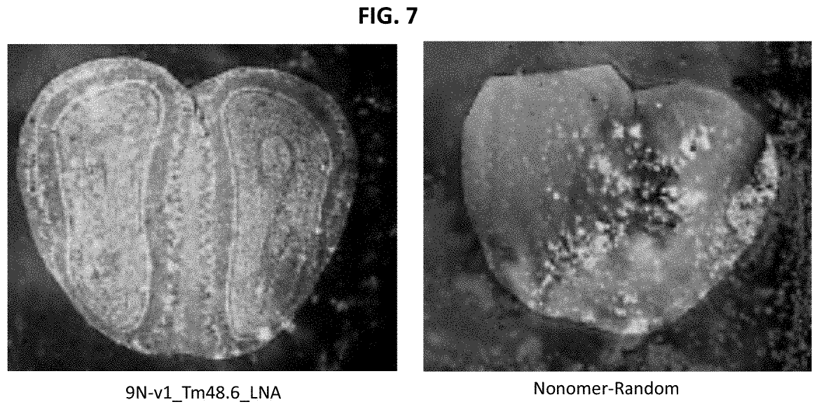

Various melting temperatures, including 48.6° C., 46.5° C., 36.5° C., and 37.4° C., were examined. As shown in FIG. 7, use of an LNA randomer capture domain on a capture probe (e.g., 9N-v1 Tm48.6 LNA, left image) at 48.6° C. hybridization temperature resulted in increased capture efficiency compared to the non-LNA nonomer randomer capture probe control (right image). These data demonstrate the efficacy in designing randomer capture probes to include LNAs. In addition to increased stability, LNAs provide the ability to modulate the melting temperature, which has the potential to increase capture efficiency.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining location of non-polyadenylated RNA in a biological sample, the method comprising:
(a) providing an array comprising a plurality of randomer capture probes, wherein a randomer capture probe comprises a spatial barcode and a capture domain sequence comprising a randomized nucleotide sequence substantially complementary to all or a portion of a sequence of the non-polyadenylated RNA;
(b) hybridizing the non-polyadenylated RNA to the capture domain of the randomer capture probe; and
(c) determining (i) all or part of the sequence of the non-polyadenylated RNA hybridized to the capture domain of the randomer capture probe or a complement thereof and (ii) the spatial barcode or a complement thereof, thereby determining the location of the non-polyadenylated RNA in the biological sample.

2. The method of claim 1, wherein the randomer capture probe is a DNA probe and the randomized nucleic acid sequence of the capture domain comprises a random hexamer sequence or a random nonomer sequence.

3. The method of claim 2, wherein the random hexamer sequence or the random nonomer sequence comprises one or more modified nucleotides.

4. The method of claim 3, wherein the modified nucleotides are locked nucleic acids.

5. The method of claim 1, wherein the randomer capture probe further comprises one or more functional domains, a unique molecular identifier, a cleavage domain, or any combination thereof.

6. The method of claim 1, wherein the non-polyadenylated RNA is a long noncoding RNA (lncRNA) molecule, a microRNA (miRNA) molecule, a small interfering RNA (siRNA) molecule, a Piwi-interacting RNA (piRNA) molecule, a small nucleolar RNA (snoRNA) molecule, a long intervening/intergenic noncoding RNAs (lincRNA) molecule, or any combination thereof.

7. The method of claim 1, further comprising hybridizing a plurality of undesirable RNA depletion probes with a plurality of undesirable RNA molecules in the biological sample, thereby generating a plurality of undesirable RNA depletion probe-undesirable RNA complexes.

8. The method of claim 7, wherein hybridizing the plurality of undesirable RNA depletion probes to the undesirable RNA molecules in the biological sample is performed between steps (a) and (b).

9. The method of claim 7, wherein the undesirable RNA molecule is a transfer RNA (tRNA), a ribosomal RNA (rRNA), a messenger RNA (mRNA), a mitochondrial RNA, a nuclear RNA, a cytoplasmic RNA, or any combination thereof.

10. The method of claim 7, further removing the plurality of undesirable RNA depletion probe-undesirable RNA complexes by contacting the biological sample with a RNase.

11. The method of claim 1, wherein the biological sample was previously stained using hematoxylin and eosin (H&E), immunofluorescence, or immunohistochemistry.

12. The method of claim 1, wherein the method further comprises permeabilizing the biological sample with a permeabilization agent selected from an organic solvent, a detergent, and an enzyme, or any combination thereof.

13. The method of claim 12, wherein the permeabilization agent is an endopeptidase or a protease.

14. The method of claim 12, wherein the permeabilization agent is pepsin or proteinase K.

15. The method of claim 1, further comprising:
extending a 3′ end of the capture domain of the randomer capture probe using the non-polyadenylated RNA as a template to generate an extended randomer capture probe; and
amplifying the extended randomer capture probe, thereby generating an amplified product comprising (i) the sequence of the randomer capture probe, or a complement thereof, (ii) all or a part of the sequence of the non-polyadenylated RNA, or a complement thereof, and (iii) the spatial barcode, or a complement thereof.

16. The method of claim 1, wherein the determining step comprises sequencing.

17. The method of claim 1, wherein the non-polyadenylated RNA molecule is associated with a disease or condition comprising an increased viral RNA in a host, an increased bacterial RNA in the host, cancer, an inflammatory disorder, a metabolic disorder, or a nervous system disorder.

18. The method of claim 1, wherein the biological sample is a tissue sample that is a fresh tissue sample, a frozen tissue sample, or a fixed tissue sample.

19. The method of claim 18, wherein the fixed tissue sample is a formalin-fixed paraffin-embedded (FFPE) tissue sample, and wherein the FFPE tissue sample is decrosslinked.

20. The method of claim 1, wherein the array further comprises a second plurality of capture probes, wherein a capture probe of the second plurality of capture probes comprises a second spatial barcode and a homopolymeric capture domain.

21. The method of claim 20, wherein the homopolymeric capture domain of the capture probe comprises a poly-thymidine sequence, and wherein the second plurality of capture probes and the randomer capture probes are distributed substantially evenly on the array.

22. The method of claim 20, wherein the homopolymeric capture domain of the second plurality of capture probes comprises a poly-thymidine sequence, and wherein;
the concentration of the second plurality of capture probes on the array is higher than the concentration of the randomer capture probes on the array, or the concentration of the second plurality of capture probes on the array is lower than the concentration of the randomer capture probes on the array.

23. The method of claim 20, further comprising:
hybridizing the poly-thymidine sequence of the capture domain of the second plurality of capture probes to a sequence corresponding to mRNA from the biological sample; and
determining (i) all or a part of the mRNA, or a complement thereof, and (ii) the second spatial barcode, or a complement thereof, and using the determined sequence of (i) and (ii) to identify the location of the mRNA in the biological sample.

24. The method of claim 23, further comprising:
after hybridizing, extending the poly-thymidine sequence of the capture domain using the hybridized sequence corresponding to the mRNA as a template, thereby generating an extended poly-thymidine capture probe; and
amplifying the extended poly-thymidine capture probe prior to determining.

25. The method of claim 20, further comprising detecting the location of a protein in a biological sample comprising:
providing a plurality of protein capture agents to the biological sample, wherein a protein capture agent of the plurality comprises:
(i) a protein binding moiety that binds specifically to the protein,
(ii) a protein binding moiety barcode, and
(iii) a protein capture sequence, wherein the protein capture sequence hybridizes specifically to the homopolymeric capture domain of the capture probe on the array;
hybridizing the protein capture sequence to the capture probe ; and
determining (i) all or a part of the protein binding moiety barcode, or a complement thereof, and (ii) the second spatial barcode of the capture probe, or a complement thereof, and using the determined sequence of (i) and (ii) to identify the location of the protein in the biological sample.

26. The method of claim 25, further comprising:
after hybridizing, extending the capture domain of the homopolymeric capture probe using the protein capture sequence as a template, thereby generating an extended capture probe; and
amplifying the extended capture probe prior to the determining step.

27. A spatial array comprising:
a plurality of randomer capture probes, wherein a randomer capture probe of the plurality of randomer capture probes comprises:
a) a capture domain comprising a random hexamer sequence or a random nonomer sequence;
b) a spatial barcode; and
a plurality of homopolymeric capture probes, wherein a capture probe of the plurality of homopolymeric capture probes comprises:
a) a capture domain comprising a poly-thymidine sequence; and
b) a spatial barcode.

28. The spatial array of claim 27, wherein each of the plurality of randomer capture probes and the plurality of homopolymeric capture probes comprises one or more functional domains, a unique molecular identifier, a cleavage domain, or any combination thereof

29. The spatial array of claim 27, wherein the capture domain of the randomer capture probe is hybridized to a non-polyadenylated RNA, and wherein the capture domain of the homopolymeric capture probe is hybridized to a sequence corresponding to an mRNA.

30. The spatial array of claim 27, wherein the plurality of homopolymeric capture probes and the plurality of randomer capture probes are distributed substantially evenly on the array.


SEQUENCE LISTING (3 sequences)

SEQ ID NO: 1-2 - skipped (2 entries)

SEQ ID NO: 3 - DNA, 14 bp, synthetic construct
  source 1..14: mol_type = other DNA
tcgactacgtagcg